(19) United States
(12) Patent Application Publication
(10) Pub. No.: US 2003/0211494 A1
(43) Pub. Date: Nov. 13, 2003

(54) Retrieval of genes and gene fragments from complex samples

(75) Inventors: Gudmundur O. Hreggvidsson, Reykjavik (IS); Olafur H. Fridjonsson, Reykjavik (IS); Sigurlaug Skirnisdottir, Reykjavik (IS); Jakob K. Kristjansson, Gardabaer (IS)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Prokaria, ltd., Reykjavik (IS)

(21) Appl. No.: 10/200,055

(22) Filed: Jul. 18, 2002

(30) Foreign Application Priority Data
May 3, 2002 (IS) 6372

(51) Int. Cl. C12Q 1/68; C12P 19/34

(52) U.S. Cl. 435/6; 435/91.2

(57) ABSTRACT

The present invention features methods of obtaining a specific DNA sequence from a complex sample. The present invention also features methods for obtaining functional genes encoding aminocyclases, amidohydrolases, and/or amylases. In addition, the invention relates to nucleic acid sequence and polypeptide sequences obtained according to the methods of the present invention.

### RELATED APPLICATION

This application claims priority under 35 U.S.C. §119 or 365 to Iceland Application No. 6372, filed May 3, 2002. The entire teachings of the above application are incorporated herein by reference.

### BACKGROUND OF THE INVENTION

The growing use of biological catalysts in the chemical synthesis, research reagent, diagnostic reagent and chemical process industries has increased the demand for the discovery and development of new enzymes. Most commercially available enzymes used today have been derived from already cultivated bacteria or fungi. The realization that less than 1% of naturally occurring microorganisms can be isolated and grown in pure culture has created great interest in developing methods to get access to uncultivated microbes in order to exploit a larger fraction of the microbial diversity than has been possible with the presently available technology. This diversity may be both in the form of unknown gene families and genetic variation within known protein families. Various strategies have been developed to access this diversity for biotechnological purposes and to pull out interesting enzyme coding genes from unculturable species. Currently, two main approaches have been used: PCR amplifications of the genes of interest and screening of shotgun libraries. The standard procedure which is based on construction and screening of DNA libraries for the genes of interest by massive sequencing, hybridizations or activity assays (expression cloning) has been widely used. These approaches can be applied on highly diverse DNA samples (Woo et al., 1994; Dalboge, 1997; Rondon et al., 1999; Short, 1999; Henne et al., 2000). Expression cloning is the only method not dependent on known sequence information. Therefore, it is likely to pull out unique sequences and complete, functional genes. However, this method is laborious and time consuming and is only made possible by high throughput laboratory methods (Dalboge, 1997; Short, 1999). Large gene libraries need to be created and screened, but full representation of “all genes” from complex environmental DNA samples is not possible because DNA from the most prevalent organisms will dominate the library and access to rare organisms cannot be achieved. Results are also dependent on the availability of good selection methods for positive clones and many factors may affect the host-donor compatibility of genes for expression. In order to obtain expression, complete genes or functional gene parts are needed, the genes have to be in the right orientation and the genes of interest need to be close to the promoter of the vector. Otherwise, low or no expression will be obtained. Furthermore, high quality DNA is a prerequisite for the library construction, i.e., it cannot contain inhibitors that may prevent the subsequent necessary restriction and ligation reactions for the clone library construction. If sequence information is used for screening such a library, i.e., by hybridization with homologous probes, the resolution of the method is dependent on similarity of the probe to the target gene. Application of polynucleotide probes may be restricted due to low homology to target genes. The application of oligonucleotide probes requires laborious standardization and may be difficult to perform in a high throughput way. Taken together, methods based on library construction have severe limitations in terms of retrieving high gene diversity from rare and uncultivated organisms in complex environmental DNA and therefore, they do not enable access to diversity in an effective way.

Different PCR approaches have also been developed to access environmental diversity and these methods have the potential to retrieve higher gene diversity than the library construction methods. It is the nature of the PCR method and the rapidly expanding sequence information available today which make the PCR approach so promising. The PCR screening procedure is similar for every gene, whereas different assay methods have to be used for different enzymes in activity screening of libraries. Conserved regions in enzyme-encoding genes serve as target sites for degenerate primers. Homology to only short sequence regions corresponding to 12-18 nucleotides is required. Thus, a set of screening primers taking into account minor sequence variation in the region for specific enzyme families can be designed. The amplification procedure can be optimized by using different buffer systems, polymerases or specially designed PCR primers. The gene specific primers can be designed in such a way that they reflect specific codon or GC bias, or contain stabilizing sequences.

Generally, PCR amplification procedure is based on the application of two specific primers. Therefore, in PCR screening, two conserved target sites with favourable length of interval sequence are required. Although, the method can be adapted in a high throughput manner to obtain gene fragments from complex environmental DNA (Radomski et al., 1998), the dependency of two conserved sequence regions in the same gene, severely limits the obtainable diversity, i.e., decreases the possibility to retrieve unknown sequences. Methods based on the use of a single gene specific primer (i.e., where the PCR amplification is dependent on one specific primer target site) have been developed, e.g., panhandle PCR (Jones and Winistorfer, 1992; Jones and Winistorfer, 1993; Megonigal et al., 2000), vectorette PCR (Riley et al., 1990; Rubie et al., 1999), dephosporylated adapters (Morris et al. 1998), oligo-cassette mediated PCR (Rosenthal and Jones, 1990; Kilstrup and Kristiansen, 2000), gene cassette PCR (Stokes et al., 2001) and bubble-cassette PCR (Laging et al., 2001). Most of theses single gene PCR methods have only been used on DNA samples from single species harbouring limited number of genes.

### SUMMARY OF THE INVENTION

In a first general aspect, the invention provides a method for obtaining at least one specific DNA sequence related to a target sequence, from a sample comprising a mixed population of a plurality of microbial species, comprising DNA or a mixture of nucleic acids, the method comprising:

a) extracting the DNA or mixture of nucleic acids from said sample;

b) hybridizing said DNA or mixture of nucleic acids with a degenerate primer targeted to a single region in said target sequence to synthesize at least one single stranded copy-DNA complementary to a region of said target sequence, said synthesis being primed by said degenerate primer and catalyzed by a DNA-polymerase or a reverse transcriptase; and performing a linear amplification of said at least one single stranded copy-DNA by repeated thermal cycling;

c) purifying the single stranded copy-DNA synthesized in step b);

d) providing a second primer site to the 3′ end of the single stranded copy-DNA; and

e) amplifying the single stranded copy-DNA using a primer pair wherein a first primer comprises at least a part of the degenerate primer sequence and a second primer which is complementary to the 3′ primer site of step d) or is an arbitrary primer;

to thereby obtain at least one specific DNA sequence related to said target sequence.

Said second primer site may be provided by a number of techniques which are described in greater detail herein. In preferred embodiments, the second primer site is provided by a method selected from the group consisting of:

ligating an anchor sequence to the 3′ end of the purified single stranded copy-DNA;

producing an anchor sequence by successively adding nucleotides to the 3′ end of the purified single stranded copy-DNA by use of terminal DNA transferase;

using an arbitrary primer;

ligating a double stranded oligonucleotide adaptor to a fragmented target DNA, following enzymatic restriction or mechanical treatment prior to generation of single stranded DNA; and

ligating fragmented targeted DNA following enzymatic restriction or mechanical treatment to vector DNA.

In another preferred embodiment, a 3′ anchor sequence is ligated to the copy-DNA by means of a ligating enzyme for ligating single stranded DNA as catalyst, such as T4 RNA ligase.

The amplification of the single stranded copy-DNA may be suitably performed by a method selected from the group of amplification methods comprising amplification methods that are dependent on a 5′ located and a 3′ located primer. Such methods include the presently preferred polymerase chain reaction (PCR) method, nucleic acid sequence based amplification (NASBA) and strand displacement amplification (SDA).

As explained in further detail herein, said degenerated primer consists in particular embodiments of a short 3′ degenerate core region and a longer 5′ consensus clamp region. The short degenerate core region will typically be in the range from about 8 to about 15 nucleotides (nt) such as, e.g., from about 9 to about 12 nt, for example 9, 10, 11 or 12 nt; whereas the longer 5′ consensus clamp region typically is in the range from about 10 to about 35 nucleotides, such as from about 12 to about 30, or from about 12 to about 29, e.g., from about 15 to about 25 nt. The CODEHOP strategy is a particularly useful method of this kind.

In presently preferred embodiments of the invention, said degenerated primer is at its 5′ end labeled with one member of an affinity pair, to allow an affinity-based purification of the linearly amplified single stranded copy-DNA. Examples of affinity pairs include but are not limited to the following: biotin—streptavidin, biotin—avidin, digoxigenin—anti-hapten antibody, fluorescein—anti-hapten antibody, lectins—lectin receptor, Ion—Ion chelators, IgG—protein A, IgG—protein G and magnets—paramagnetic particles. A particularly preferred affinity binding pair is the biotin-streptavidin pair.

As will be appreciated by the skilled person, the DNA sequences obtained by the present invention may be used to retrieve functional genes comprising said sequences. Consequently, the method of the invention comprises in one embodiment steps of amplifying flanking regions to the obtained DNA sequence to obtain a functional gene comprising said DNA sequence. Said flanking regions may for example be amplified with one or more steps of nested PCR reactions, such as demonstrated in Example 5 herein.

In another alternative embodiment, the method comprises the step of screening said sample to isolate a functional gene encoding a protein, using a probe having a sequence which is the same as or complementary to at least a portion of said obtained DNA sequence.

As described above, among the surprising aspects of the present invention is the ability to retrieve genes from highly complex samples. In one embodiment, said sample of DNA or nucleic acids is a complex mixture of nucleic acids extracted from mixed cultures of microorganisms. In certain useful embodiments, said sample of DNA or nucleic acids is a complex mixture of nucleic acids extracted from an environmental sample. Examples of environmental samples include but are not limited to samples derived from oligotrophic environments, extreme environments, (e.g., a terrestrial geothermal environment such as a hot spring, or hot soil), and a marine geothermal environment.

In yet another embodiment of the method as described herein, the sample is enriched for a microbial population by maintaining the sample under conditions substantially similar to the environment from which the sample was obtained to thereby expand the microbial population; and allowing a sufficient quantity of a microbial population to expand; whereby the population has been enriched.

The invention also pertains to a method for obtaining a functional gene encoding an aminoacylase/amidohydrolase from a sample comprising DNA and/or a mixture of nucleic acids (such as, e.g., a sample comprising complex DNA as described above), comprising screening said sample using as a probe a nucleic acid comprising a nucleotide sequence which is selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:28, SEQ ID NO:29, SEQ ID NO:30, SEQ ID NO:31, sequences which hybridize to said sequences under stringent conditions, and sequences encoding for polypeptides having at least 75% sequence identity but preferably higher such as e.g., at least 80% or at least 85%, and more preferably at least 90%, including at least 95% or at least 97% sequence identity to polypeptides encoded for by any of the sequences of SEQ ID NOs:1-9 or SEQ ID NOs:28-31, and sequences encoding for polypeptides having at least 65% sequence identity and preferably 70% sequence identity to polypeptides encoded for by any of the sequences of SEQ ID NOs: 1-9 or SEQ ID NOs:28-31, and complementary sequences thereto.

In a further aspect, the invention provides a method for obtaining a functional gene encoding an amylase from a sample comprising DNA and/or a mixture of nucleic acids, comprising screening said sample using as a probe a nucleic acid comprising a nucleotide sequence from the group consisting of SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:20, SEQ ID NO:21, SEQ ID NO:22, SEQ ID NO:23, SEQ ID NO:24, SEQ ID NO:25, SEQ ID NO:26, SEQ ID NO:27, sequences which hybridize to said sequences under stringent conditions, and sequences encoding for polypeptides having at least 65% and preferably at least 70% sequence identity but more preferably higher identity such as e.g., at least 80% or at least 90% sequence identity including at least 95% or at least 97% sequence identity to polypeptides encoded for by any of said sequences, and complementary sequences thereto.

Yet a further aspect of the invention pertains to a method for obtaining a functional gene encoding an amylase from a sample comprising DNA and/or a mixture of nucleic acids comprising the step of screening said sample using a nucleic acid probe comprising a nucleotide sequence from the group of SEQ ID NO:19, sequences encoding for polypeptides having at least 80% sequence identity and preferably at least 90% or at least 95% including at least 97% or at least 99% sequence identity to a polypeptide encoded for by the sequence of SEQ ID NO: 19, for example, SEQ ID NO: 60, and complementary sequences thereto.

Several novel gene fragments and gene sequences have been identified and obtained by use of the present invention. These sequences belong to the aminoacylase/amidohydrolase protein family and amylase protein family, cf. Tables 2-7 sequences.

Consequently, in a further aspect of the invention, an isolated nucleic acid molecule is provided, having a nucleic acid sequence which is part of a gene encoding for an aminoacylase/amidohydrolase, said sequence being selected from the group consisting of SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9, SEQ ID NO:28; SEQ ID NO:29; SEQ ID NO:30; and SEQ ID NO:31, and sequences encoding a polypeptide having at least 75% sequence identity, and preferably higher identity such as at least 80% sequence identity and more preferably at least 90% sequence identity such as at least 95% sequence identity, including at least 97% or 99% sequence identity with a polypeptide encoded for by any of the sequences SEQ ID NOs: 1-9 or SEQ ID NOs: 28-31, and sequences encoding for polypeptides having at least 65% sequence identity and preferably 70% sequence identity to polypeptides encoded for by any of said sequences SEQ ID NOs: 1-9 or SEQ ID NOs: 28-31. Also provided is an isolated nucleic acid having a sequence encoding for an aminoacylase/amidohydrolase, said nucleic acid comprising a nucleic acid sequence as described above.

Also provided herein is an isolated nucleic acid molecule having a nucleic acid sequence which is part of a gene encoding for an amylase, said sequence being selected from the group consisting of SEQ ID NO:10, SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19, SEQ ID NO:20; SEQ ID NO:21; SEQ ID NO:22; SEQ ID NO:23; SEQ ID NO:24; SEQ ID NO:25; SEQ ID NO:26; SEQ ID NO:27, and sequences encoding a polypeptide having at least 65% and preferably at least 70% sequence identity, and more preferably higher identity such as at least 80% sequence identity and more preferably at least 90% sequence identity such as at least 95% sequence identity, including at least 97% or at least 99% sequence identity with a polypeptide encoded for by any of the sequences SEQ ID NOs: 10-18 or SEQ ID NOs: 20-27. Also provided is an isolated nucleic acid having a sequence encoding for an aminoacylase/amidohydrolase, said nucleic acid comprising a nucleic acid sequence as described above.

In a yet further aspect an isolated nucleic acid molecule having a sequence encoding for an amylase is provided, which nucleic acid comprises one of the above described nucleic acid sequences that are part of amylase encoding genes.

In a still further aspect, an isolated polypeptide is provided (i.e., an aminoacylase/amidohydrolase, or an amylase) encoded by any of above described nucleotide sequences. In particular embodiments, the invention provides isolated polypeptides comprising a sequence from the group of SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:69, SEQ ID NO:70, SEQ ID NO:71, and SEQ ID NO:72, SEQ ID SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:61, SEQ ID NO:62, SEQ ID NO:63, SEQ ID NO:64, SEQ ID NO:65, SEQ ID NO:66, SEQ ID NO:67, and SEQ ID NO:68.

Such polypeptides may be readily cloned and overexpressed by well-known methods based on the information provided herein.

### BRIEF DESCRIPTION OF THE DRAWINGS

FIG. 1 is a schematic representation of the method of the present invention, wherein an adaptor sequence is ligated to the 3′ end of the single stranded copy-DNA to provide a second primer site for the second amplification step.

FIG. 2 is a schematic representation of the method of the present invention, wherein arbitrary priming is used in the second step for the second primer site.

### DETAILED DESCRIPTION OF THE INVENTION

The invention described herein introduces and adapts several methods that have been used for amplifying genes or gene fragments from non-complex DNA and combines these methods in a new manner to enable the amplification of a number of diverse gene fragments encoding for proteins from specific protein families from highly complex DNA such as extracts from mixed cultures, enrichments and environmental samples. The invention described herein makes it possible to retrieve genes from complex samples without creating large gene libraries and using very time consuming techniques of expression screening, massive shot gun sequencing or hybridizations. We have used this technique to isolate multitude of gene fragments and complete genes of novel enzymes from mixed DNA extracted from environmental hot spring microbial biomass samples. We demonstrate in the examples how gene fragments coding for proteins within the same protein family can be isolated from complex DNA via PCR when only one block of conserved amino acid region is available.

The method of the present invention is based on using only one degenerated gene specific primer against conserved regions derived from the analysis of multiple alignments of proteins belonging to a particular protein family. It differs from prior art methods, in which the use of single gene specific primers have only been described for the purpose of isolation of unknown sequences in a single genome DNA or genome library DNA. Furthermore, in the present method one polymerase reaction takes place as the first step, wherein single-stranded polynucleotides are produced. Since no restriction or ligation of the source DNA takes place, the demands for high quality DNA are not as stringent as for the library-based methods.

The term “protein family” in this context is to be understood as comprising proteins that share sequence, structural, or functional characteristics, such as sequence similarity, conserved sequence motifs, structural domains, structural folds, or functionalities such as active sites including binding sites. Preferably, such shared characteristics are reflected by homology of the genes encoding the family proteins, such that proteins family members may be found and selected by the methods as described herein. The term “homology” and “homologous” as used herein refer generally to sequences that share sequence similarity by virtue of common descent.

The classifying term amylase refers herein generally to a group of closely related enzymes that degrade polysaccharides, specifically that are able to hydrolyse O-glucosyl linkages in starch, glycogen, and related polysaccharides. This group (“amylase family”) is also referred to as family 13 glycosyl hydrolases. Classification of glycohydrolases is based on sequence similarity and they share the same structural folds. Enzymes of the family 13 of the glycosyl hydrolases have a structure consisting of an 8 stranded alpha/beta barrel containing the active site, often interrupted by a calcium-binding domain of about 70 amino acids protruding between beta strand 3 and alpha helix 3, and a carboxyl-terminal greek key beta-barrel domain. Enzymes belonging to this family degrade or modify polysaccharides, specifically starch and glycogen, pullulan and related substrates, acting on alpha 1-4 O-glucosyl linkages with a retaining mechanism of action.

Glycoside hydrolase family 13 (CAZy GH_{—}13) comprises enzymes with a variety of known activities; alpha-amylase (EC 3.2.1.1); pullulanase (EC 3.2.1.41); cyclomaltodextrin glucanotransferase (EC 2.4.1.19); cyclomaltodextrinase (EC 3.2.1.54); trehalose-6-phosphate hydrolase (EC 3.2.1.93); oligo-alpha-glucosidase (EC 3.2.1.10); maltogenic amylase (EC 3.2.1.133); neopullulanase (EC 3.2.1.135); alpha-glucosidase (EC 3.2.1.20); maltotetraose-forming alpha-amylase (EC 3.2.1.60); isoamylase (EC 3.2.1.68); glucodextranase (EC 3.2.1.70); maltohexaose-forming alpha-amylase (EC 3.2.1.98); branching enzyme (EC 2.4.1.18); trehalose synthase (EC 5.4.99.16); 4-alpha-glucanotransferase (EC 2.4.1.25); maltopentaose-forming alpha-amylase (EC 3.2.1.-); amylosucrase (EC 2.4.1.4); sucrose phosphorylase (EC 2.4.1.7).

The terms aminoacylase (EC 3.5.1.14) and amidohydrolase (e.g., EC 3.5.1.32) refer to enzymes that catalyze any reaction of the type:

N-acyl-amino acid+H₂O->fatty acid (anion)+amino acid

These enzymes belong to the peptidase family M40. This family includes a range of zinc metallopeptidases belonging to several families in the peptidase classification.

“Stringency conditions” for hybridization is a term of art which refers to the incubation and wash conditions, e.g., conditions of temperature and buffer concentration, which permit hybridization of a particular nucleic acid to a second nucleic acid; the first nucleic acid may be perfectly (i.e., 100%) complementary to the second, or the first and second may share some degree of complementarity which is less than perfect (e.g., 60%, 75%, 85%, 95%). For example, certain high stringency conditions can be used which distinguish perfectly complementary nucleic acids from those of less complementarity.

“High stringency conditions”, “moderate stringency conditions” and “low stringency conditions” for nucleic acid hybridizations are explained on pages 2.10.1-2.10.16 and pages 6.3.1-6 in Current Protocols in Molecular Biology (Ausubel, F. M. et al., “Current Protocols in Molecular Biology”, John Wiley & Sons, (1998)) the teachings of which are hereby incorporated by reference. The exact conditions which determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, high, moderate or low stringency conditions can be determined empirically.

By varying hybridization conditions from a level of stringency at which no hybridization occurs to a level at which hybridization is first observed, conditions which will allow a given sequence to hybridize (e.g., selectively) with the most similar sequences in the sample can be determined.

Exemplary conditions are described in Krause, M. H. and S. A. Aaronson, Methods in Enzymology, 200:546-556 (1991). Also, in, Ausubel, et al., “Current Protocols in Molecular Biology”, John Wiley & Sons, (1998), which describes the determination of washing conditions for moderate or low stringency conditions. Washing is the step in which conditions are usually set so as to determine a minimum level of complementarity of the hybrids. Generally, starting from the lowest temperature at which only homologous hybridization occurs, each degree (° C.) by which the final wash temperature is reduced (holding SSC concentration constant) allows an increase by 1% in the maximum extent of mismatching among the sequences that hybridize. Generally, doubling the concentration of SSC results in an increase in Tm of about 17° C. Using these guidelines, the washing temperature can be determined empirically for high, moderate or low stringency, depending on the level of mismatch sought.

For example, a low stringency wash can comprise washing in a solution containing 0.2×SSC/0.1% SDS for 10 min at room temperature; a moderate stringency wash can comprise washing in a pre-warmed solution (42° C.) solution containing 0.2×SSC/0.1% SDS for 15 min at 42° C.; and a high stringency wash can comprise washing in pre-warmed (68° C.) solution containing 0.1×SSC/0.1%SDS for 15 min at 68° C. Furthermore, washes can be performed repeatedly or sequentially to obtain a desired result as known in the art.

The gene specific primer is degenerate for a highly conserved amino acid sequence region, which is identified by analyzing multiple alignments of proteins from the protein family that is targeted. The degenerate gene specific primer can be designed by a number of methods, including the CODEHOP method (Consensus-Degenerate Hybrid Oligonucleotide Primer) (Rose et al., 1998). The target region of the protein family being targeted should preferably contain at least 3-4 conserved amino acids.

In an embodiment of the invention, the designed gene specific primers are affinity-labelled at the 5′end (such as preferably labelled with biotin), which allows the separation of the first single stranded DNA product from the complex DNA by allowing the biotin-labelled primers to bind to streptavidin beads. After several copies of the single stranded DNA have been produced by linear amplification, a second reverse priming site can be made available by various means, such as for example, by ligating a single stranded oligonucleotide of known sequence to the 3′ end of the single stranded DNA by means of a ligase, which may suitably by a single strand-DNA ligating enzyme such as in particular T4 RNA ligase. Further, a terminal transferase can be used to add nucleotides to the 3′ end of the single stranded DNA in a tailing reaction. The modified templates are then re-amplified by using the gene specific primer (unlabelled) and a reverse primer complementing the adapter sequence primer or transferase-generated tail to make double-stranded DNA that can then be amplified by PCR for further cloning and/or sequencing. An arbitrary primer can also be used against the unlabelled gene specific primer for the re-amplification. The term “arbitrary primer” refers herein generally to a short oligonucleotide primer (such as from about 10 to about 30 nt) intended to initiate DNA synthesis at random locations on the target DNA. Such a primer will hybridize to a complementary site downstream of the first priming site that was used for the generation of the single stranded DNA. This arbitrary primer can be specifically designed with different level of degeneracy, length and nucleotide composition. The original gene specific primer (unlabelled) can also serve as an arbitrary primer. Thus, the degenerate specific primer can function both as a specific primer and an arbitrary primer in the same amplification reaction.

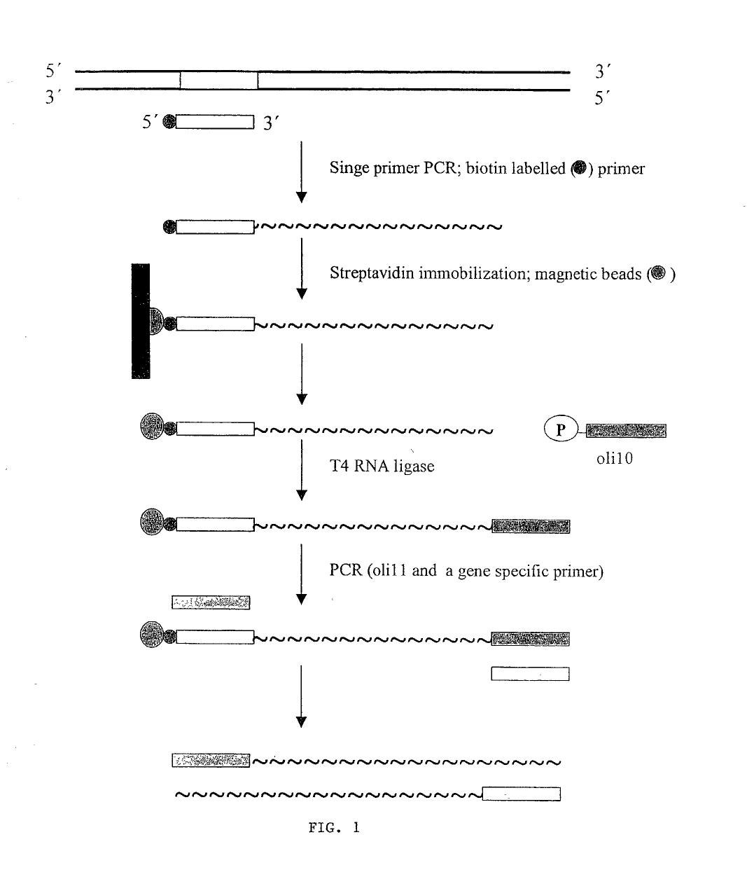

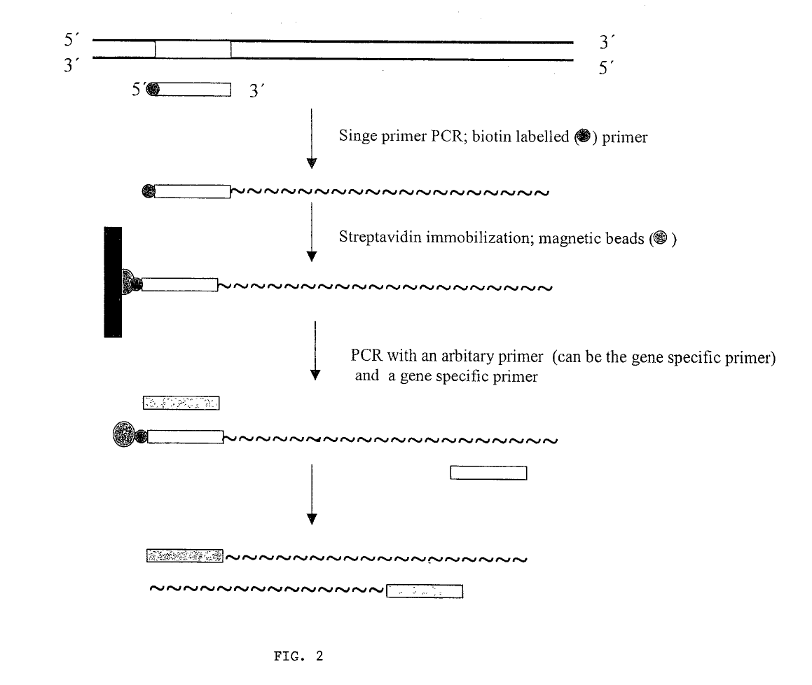

The gene fragments so obtained will provide further specific sequence information needed for the retrieval and amplification of complete genes from the original DNA mixtures extracted from the biomass or enrichment samples. The strategy for the generation of the first single-stranded fragments and for two variations of the subsequent generation and amplification of the double-stranded DNA by the present invention is illustrated in FIG. 1 and FIG. 2.

As mentioned above, a preferred embodiment of the invention uses the CODEHOP method (Consensus-Degenerate Hybrid Oligonucleotide Primer) (Rose et al., 1998)) for designing primers for generating and amplifying the single stranded fragments from distantly related sequences in the complex DNA. The primers are targeted to a conserved region in the sequences of a particular protein family of interest and consist of two regions, one short 3′-end degenerate core region and one longer 5′-end consensus clamp region. Only three or four highly conserved amino acids residues are needed for the design of the core. Preferably, a moderately conserved amino acid region upstream of the conserved amino acid residues is used for the clamp region, but arbitrary and/or specific DNA of known sequences can also be used. The core will ensure specificity and the clamp will enhance this specificity by enabling the use of higher annealing temperatures in the PCR. Reducing the length of the 3′ core to a minimum of 3 amino acids decreases the total number of individual primers in the degenerate primer pool. The 5′ non-degenerate consensus clamp stabilizes hybridization of the 3′ degenerate core with the target template.

The method of the invention described herein was tested for the retrieval of gene fragments followed by retrieving their flanking sequences to obtain complete enzyme-coding genes of starch-modifying enzymes belonging to glycoside hydrolase family 13 (here referred to as family 13 or amylase family) (Antranikian, 1990; Henrissat and Davies, 1997) and of enzymes belonging to the bacterial metal peptidase family M40, containing enzymes such as aminoacylases (E.C. 3.5.1.14) and amidohydrolases (E.C. 3.5.1.32) (here referred to as peptidase family M40 or aminoacylases/amidohydrolases) (Anders and Dekant, 1994; Rawlings and Barrett, 1995). Family 13 includes many types of different starch-modifying and starch-hydrolyzing enzymes. These enzymes include α-amylases, glycogenases, pullulanases, cyclodextrinases, 1,6 glucosidases, branching and debranching enzymes and glucanotransferases. More than one type of these enzymes is found in many bacterial and archaeal species and they can either be intracellular or extracellular. Despite different activities of the enzymes, two regions are known to be well conserved in the primary structures of these proteins.

For the purpose of comparing and demonstrating the improvements offered by the present invention over traditional methods, we also used the PCR techniques with two degenerate gene specific primers for retrieval of gene fragments belonging to glycosidase family 13 from one environmental DNA sample (see Example 1). We also demonstrate different embodiments of the single primer method for retrieval of gene fragments from two protein families, glycosidase family 13 and peptidase family M40, from environmental DNA. A total of 10 new very diverse amylase genes were isolated belonging to family 13 from a single sample using the single primer and an adaptor ligation approach, where in a parallel experiment only 4 were found using the two primer method. Three very different aminoacylase/amidohydrolase sequences were retrieved from two environmental samples by using the adaptor ligation approach in the second step of the invention, and by using the arbitrary primer approach in the second step additional 11 more diverse and highly divergent different aminoacylase/amidohydrolase sequences, were retrieved.

This demonstrates that the present invention is applicable for the retrieval of very diverse genes encoding for enzymes in different protein families. The advantages of the present invention above the state of the art were well demonstrated, as the single primer method generated far greater diversity than the conventional two gene specific primer method in parallel gene retrieval experiment of glycosidehydrolase family 13 gene fragments from the same environmental DNA sample. The gene fragments obtained from biomass samples by the present invention or variation of this invention can be used for various purposes. The obtained fragments can be used as templates in inverse PCR for retrieving flanking sequences to isolate complete genes by the use of nested primers. (see, e.g., applicant's co-pending U.S. patent application Ser. No. 09/878,423 filed on Jun. 11, 2001, “Method of Obtaining Protein Diversity”, the teachings of which are incorporated herein in their entirety). Further, the gene fragments can replace homologous fragments in recombinant host genes to construct hybrid enzymes. The fragments can further be used as nucleic acid probes to screen DNA libraries prepared from environmental DNA for the purpose of identifying and isolating the corresponding or related complete genes. Moreover, they can be used in in vitro protein evolution experiments such as input in gene shuffling to obtain enzymes with improved properties, that can subsequently be modified by mutational treatment such as with error prone PCR methods.

The methodology of the present invention makes a successful link between bioinformatics and bioprospecting. The method combines in a new way data-mining of the already accumulated DNA and protein sequence information, which provides a basis for retrieving unknown gene sequences and gene fragments from environmental samples without cloning. The method is simple and fast and by using highly degenerated primers, it can be used to detect and retrieve novel genes from very complex DNA from mixed cultures, enrichments and environmetal samples, including but not limited to oligotrophic and exteme environments such as hot springs (terrestrial and marine), hot soil, etc. In the invented gene retrieval method we use successive PCR amplifications for first obtaining the initial gene fragment sequences, followed by the retrieval of complete genes directly from biomass DNA. In the first amplification, we use one degenerated gene specific primer designed for a conserved site that is determined from analysis of multiple alignments of known sequences, as described above. The second reverse primer, or a second reverse primer site for retrieval and amplification of double stranded DNA gene fragments, can be supplied by various means as described as above.

The second reverse priming site can also be supplied to the template DNA prior to the PCR by several known methods such as by first fragmenting the environmental DNA either by restriction or mechanically followed by ligating a double stranded oligonucleotide adapter. To prevent unspecific amplification by the reverse primer from the adapters ligated to both ends of the DNA fragments various methods can be used, such as using dephosphorylated adapters so that ligation takes only place to the 5′ primer end of the sample DNA fragments (Morris et al 1998) oligo-cassettes (Rosenthal and Jones, 1990; Kilstrup and Kristiansen, 2000), gene cassette PCR (Stokes et al., 2001) and bubble-cassette PCR (Laging et al., 2001). Another embodiment of the invented method involves supplying the second priming site by a vector. The sample DNA is fragmented and cloned into a vector that can be a plasmid or a phage prepared in such a way that it has a single unique priming site bordering one side of the insert that can then be used as the second reverse priming site (Shyamala and Ames, 1989).

As mentioned above, it is found particularly useful to use the methods of the present invention for samples that have been enriched for a microbial population. Such enrichment strategies are described in detail in applicant's co-pending application (U.S. patent application Ser. No. 09/770,771 “Accessing Microbial Diversity by Ecological Methods”, which is hereby incorporated by reference in its entirety; see also PCT/IS02/00003). With such methods, different fractions of microbial populations may be enriched from natural environments with variable diversity, depending on substrate and physiochemical conditions. The methods may comprise enriching the environmental conditions with a chemical additive (e.g., nutrient, mineral, salt, etc.). The term enrichment in this context is meant to indicate the act of increasing the proportion of one or more desired species by introducing nutrients and/or conditions or solid support required for increasing the population of the species of interest.

Novel Nucleotide Sequences and Polypeptides of the Invention

As mentioned above, several novel gene fragments and gene sequences have been identified and obtained by use of the present invention. These sequences belong to the aminoacylase/amidohydrolase protein family and amylase protein family, cf. Tables 2-7 sequences. The sequences are particularly useful for obtaining functional genes encoding novel aminoacylase/amidohydrolases and amylases, such as by use of the methods described herein.

The novel nucleotide sequences and corresponding isolated nucleic acid molecules provided by the present invention that are parts of genes encoding aminoacylase/amidohydrolases are listed and described in Tables 2 and 3 and depicted as SEQ ID NOs: 1-9 and SEQ ID NOs: 28-31.

Similarly, nucleotide sequences and corresponding isolated nucleic acid molecules that are parts of genes encoding amylases are listed and described in Tables 4-6 and depicted as SEQ ID NOs: 10-27.

Isolated nucleic acid molecules comprising functional genes that comprise the above-mentioned nucleotide sequences are readily obtainable by well-known methods, for example, by obtaining the flanking regions of the obtained sequences by a series of nested PCR reactions, e.g., as described in detail in Example 5. Consequently, such isolated nucleic acid molecules comprising any of the above-mentioned sequences and related sequences as described above are also provided by the invention. Preferably, such isolated nucleic acid molecules comprise functional genes encoding polypeptides with any of said activities.

The invention further relates to isolated polypeptides obtainable by cloning and overexpression of the nucleic acid molecules provided by the invention. Preferred polypeptides of the invention comprise a sequence selected from the sequences depicted as SEQ ID NOs: 42-72. The polypeptides may be partially or substantially purified (e.g., purified to homogeneity) and/or substantially free of other polypeptides. According to the invention, the amino acid of the polypeptide can be that of the naturally occurring polypeptide or can comprise alterations therein. Polypeptides comprising alterations are referred to herein as “derivatives” of the native polypeptide. Such alterations include conservative or non-conservative amino acid substitutions, additions and deletions of one or more amino acids; however, such alterations should preserve at least one activity of the polypeptide, i.e., the altered or mutant polypeptide should be an active derivative of the naturally occurring polypeptide.

Additionally included herein are active fragments of the polypeptides described herein, as well as fragments of the active derivatives described above. An “active fragment,” as referred to herein, is a portion of a polypeptide (or a portion of an active derivative) that retains the polypeptide's activity, as described above. Included in the invention are polypeptides which have at least about 90% or at least about 95%, at least about 97% sequence identity to the polypeptides described herein (i.e., the polypeptides encoded for by the genes and gene fragments described herein). However, polypeptides exhibiting lower levels of identity are also useful, such as those having at least about 65% sequence identity or at least about 70% sequence identity, and more preferably at least about 75% or at least about 80% sequence identity to the polypeptides described herein, particularly if they exhibit high (e.g., at least about 90% or at least about 95%) sequence identity to one or more particular domains of the polypeptide, e.g., the active site domain.

The polypeptides may be recombinantly produced. For example, PCR primers can be designed (e.g., by use of the nucleic acid sequences provided herein) to amplify the encoding genes. The primers can contain suitable restriction sites for efficient cloning into a suitable expression vector. The PCR product can be digested with the appropriate restriction enzyme and ligated between the corresponding restriction sites in the vector. The polypeptides of the present invention can be isolated or purified (e.g., to homogeneity) from cell culture (e.g., from culture of host cells comprising the expression vector) by a variety of processes. These include, but are not limited to anion or cation exchange chromatography, ethanol precipitation, affinity chromatography, and high performance liquid chromatography (HPLC). The particular method used will depend upon the properties of the polypeptide; appropriate methods will be readily apparent to the person skilled in the art.

To determine the percent identity of two nucleic acid sequences, the sequences can be aligned for optimal comparison purposes (e.g., gaps can be introduced in the sequence of a first nucleotide sequence). The nucleotides at corresponding nucleotide positions can then be compared. When a position in the first sequence is occupied by the same nucleotide as the corresponding position in the second sequence, then the molecules are identical at that position. The percent identity between the two sequences is a function of the number of identical positions shared by the sequences (i.e., % identity=# of identical positions/total # of positions×100).

The determination of percent identity between two sequences can be accomplished using a mathematical algorithm. A preferred, non-limiting example of a mathematical algorithm utilized for the comparison of two sequences is the algorithm of Karlin et a.l (1993). Such an algorithm is incorporated into the NBLAST program which can be used to identify sequences having the desired identity to nucleotide sequences of the invention. To obtain gapped alignments for comparison purposes, Gapped BLAST can be utilized as described in Altschul et al. (1997). When utilizing BLAST and Gapped BLAST programs, the default parameters of the respective programs (e.g., NBLAST) can be used. In one embodiment, parameters for sequence comparison can be set at W=12. Parameters can also be varied (e.g., W=5 or W=20). The value “W” determines how many continuous nucleotides must be identical for the program to identify two sequences as containing regions of identity.

The invention is further illustrated by the Examples which are not intended to be limiting in any way. All references cited herein are incorporated herein by reference in their entirety.

### EXAMPLES

### Example 1

### Sample Collection and DNA Extraction

Three different environmental and enrichment biomass and water samples were collected and used for preparation of source DNA. Sample Z contained water plus microbial mat biomass and was collected from a basin of a hot spring at 80° C. and at pH 8.5. Sample 173 contained sediment plus microbial biomass from a hot spring at 67° C. and pH 8.0 and sample 202B contained soil plus fluid from an in situ sponge support enrichment incubated for 3 weeks in a hot soil location at 92° C. and pH 6.0. In order to separate the microorganisms from other particles in the samples, the samples were vigorously mixed with water and shaken in a stomacher before the DNA was extracted. Genomic DNA from the above environmental biomass samples was extracted as described by Marteinsson et al. 2001 (Marteinsson et al., 2001b).

16S rRNA Analysis

To determine the quality and complexity of the environmental DNA, a library of bacterial 16S rRNA genes was prepared from the DNA from of samples Z, 173 and 202B. Molecular diversity analysis was done on the DNA as described earlier (Skirnisdottir et al., 2000).

A total of 49, 62 and 135 clones were analysed for samples 202B, Z and 173 respectively. Table 1 shows the frequencies and the phylogenetic position of the 16S rRNA sequences obtained from the environmental biomass DNA samples. A similarity of 98% was used as a cut-off value for grouping the sequences into different operational taxonomic units (OTUs) (Skirnisdottir et al., 2000). The degree of diversity in all samples was high, as shown in Table 1. Samples 202B, 173 and Z gave 31, 25 and 14 OTUs, respectively.

### Example 2

### Retrieval of Gene Fragments Coding for Enzymes Belonging to Peptidase Family M40, Using Single Gene Specific Primer in the First Step and Adapter-Supplied Priming Site in the Second Step

Samples

Samples 173 and 202B from Example 1 were used as source DNA.

Construction of Degenerated Primers

For the primer construction, amino acid sequences of various aminoacylase/amidohydrolase enzymes were retrieved from protein databases (Bateman et al., 1999; Maidak et al., 1999) and aligned by using CLUSTALX version 1.8. (Thompson et al., 1997). Furthermore, blocks of multiply aligned amino acid sequences, established with the program Blockmaker (Henikoff et al., 1995) were used as input for the CODEHOP program. Primers were designed according to the CODEHOP strategy by using the CODEHOP program (Rose et al., 1998). The primers were degenerate at the 3′ core region of length 11 bp across four codons of highly conserved amino acids. In contrast, they were non-degenerate at the 5′ region (consensus clamp region) of 12 and 16 bp with the most probable nucleotide predicted for each position. Two different reverse primers of the same region were made for the aminoacylase/amidohydrolase screening. The primers were AA3 (5′-CATTGCCGTATGGCCAtcrtgnccrca-3′; degeneracy 16: reverse) (SEQ ID NO: 32) and AA4 (5′-GGCCGTGTGGCCtcrtgnccrca-3′; degeneracy 16: reverse) (SEQ ID NO: 33). Letters in lower case correspond to the core region and upper case letters correspond to the consensus clamp region.

Linear PCR with Single Degenerate Family Specific Primer

The DNA from samples 173 and 202B were used as templates for aminoacylase/amidohydrolase gene-specific primers AA3 and AA4. The primers were biotin labelled at the 5′ end (MWG Biotech, Ebersberg, Germany). The PCR was carried out in 50 μl reaction mixture containing 1-100 ng of genomic DNA (dilutions used), 0.2 μM AA3 or AA4, 200 μM of each dNTP in 1× DyNAzyme DNA polymerase buffer and 2.0 U DyNAzyme DNA polymerase (Finnzymes) with a MJ Research thermal cycler PTC-0225. The reaction mixture was first denatured at 95° C. for 5 min, followed by 40 cycles of denaturing at 95° C. (50 s), annealing at five different temperatures (40° C., 43.8° C., 50° C., 57.3° C. and 62° C.) for 50 s and extension at 72° C. (2 min). Samples were loaded on 1% a TAE agarose gel to identify unspecific priming. Those samples giving no visible bands, from the different annealing temperatures for each primer, thus indicating low unspecific priming, were selected for re-amplification and were pooled prior to the QIAGEN PCR purification step.

PCR Purification and Immobilization of Single Stranded PCR Products

To remove excess of biotin labelled primers, nucleotides and polymerase, the PCR samples were passed through QIAquick PCR purification spin columns (QIAGEN, Germany) by following the manufacturers instructions. The samples were eluted with 30 μl of H₂O and then the biotin labelled PCR products were immobilized by using 150 μg of streptavidin-coated magnetic beads (Dynal, Oslo, Norway) according to the instructions of the manufacturer. The captured biotin labelled PCR products were resuspended in 11 μl of dH₂O. PCR products from the different annealing temperatures for each primer of the aminoacylase/amidohydrolase genes were pooled in the QIAGEN PCR purification step. The immobilized single stranded DNA was then subjected to a ligation reaction as described below.

Ligation of an Adaptor (oli10) to the Single Stranded Biotin Labelled PCR Products Using T4 RNA Ligase

In the presence of 20 U of T4 RNA ligase (New England BioLabs, Beverly, Mass., USA), T4 RNA ligation buffer (50 mM Tris-HCl, pH 7.8, 10 mM MgCl 2, 10 mM DTT and 1 mM ATP) and 10% PEG8000, 50 nM of the adaptor 5′-phosphorylated oligodeoxyribonucleotide oli10 (5′-AAGGGTGCCAACCTCTTCAAGGG-3′; oli10 in FIG. 1) (SEQ ID NO: 34) was added to the captured DNA in a final volume of 20 ill. The mixture was incubated at 22° C. for 24-60 h.

Re-Amplification PCR from the Ligation Reaction

The exponential re-amplification PCR was carried out in 50 μl reaction mixture containing 2 μl ligation mixture, 1.0 μM unlabelled gene specific primer, AA3 or AA4, (the gene specific primer corresponding to the first linear PCR step), 1.0 μM oli11 (5′-CTTGAAGAGGTTGGCACCCT-3′) (SEQ ID NO: 35) which is complementary to oli10, 200 μM of each dNTP in 1× DyNAzyme DNA polymerase buffer and 2.0 U DyNAzyme DNA polymerase (Finnzymes, Espoo, Finland) with a MJ Research thermal cycler PTC-0225. The reaction mixture was first carried out by denaturing at 95° C. for 5 min, followed by 30 cycles of denaturing at 95° C. (0:50 min), annealing at 55° C. for 50 s and extension at 72° C. (2 min). This was then followed with a final extension for 7 min at 72° C. to obtain ‘A’ overhangs.

Analyzing, Purification and Cloning of the PCR Products

Seven microliters of the PCR reamplification products were taken for 1% TAE agarose gel electrophoresis to confirm the identity of the PCR products and the patterns compared between the control PCRs (gene specific primers) and the main PCRs (oli11/gene specific primers). Before cloning, thirty microliters of the PCR products were loaded on thick 1% TAE agarose electrophoresis gels. Visible re-amplification DNA products (obtained from pooled samples) of 0.2-0.5 kb were observed on agarose gels for both primers (AA3 and AA4). The bands were purified by using spin columns, GFX PCR DNA and Gel Band Purification kit according to the manufacturer (Amersham Biosciences, Hørsholm, Denmark). The samples were eluted with 25 μl of H₂O. Then the purified PCR products (4 μl) were cloned by the TA cloning method (Zhou and Gomez-Sanchez, 2000). Plasmid DNAs from single colonies were isolated and purified by using Multiscreen Separation System according to the instructions of the manufacturer (Millipore Corporation, Bedford, Mass.). Inserts in approximately 360 clones were sequenced. The gene inserts were sequenced with M13 reverse and M13 forward primers on ABI 3700 DNA sequencers by using a BigDye terminator cycle sequencing ready reaction kit according to the instructions of the manufacturer (PE Applied Biosystems, Foster City, Calif.). All sequences were analysed in Sequencer 4.0 for Windows (Gene Codes Cooperation, Ann Arbor, Mich.) and XBLAST searched (Altschul et al., 1990; Altschul et al., 1997). All sequences were imported into the program BioEdit version 5.0.6 (Tom Hall, North Carolina State University, Department of Microbiology) and aligned therein by ClustalW. Six (2%) of the 360 clone sequences gave closest hit to aminoacylase/amidohydrolase sequences, belonging to 3 different aminoacylase/amidohydrolase genes (Table 2 & 7). Aminoacylase EAA1 was found in sample 202B but the other two in sample 173.

### Example 3

### Retrieval of Gene Fragments Coding for Enzymes Belonging to Peptidase Family M40, Using Single Gene Specific Forward Primer in the First Step and Reverse Arbitrary Priming in the Second Step

Samples

Samples 173 and 202B from Example 1 were used as source DNA.

Construction of Degenerated Primers

The primer construction was as described in Example 2.

Linear PCR with Single Degenerate Family Specific Primer

The procedure for the linear PCR with the single degenerate family specific primers AA3 or AA4 was as described in Example 2.

PCR Purification and Immobilization of Single Stranded PCR Products

The purification and immobilization of single-stranded PCR products was as described in Example 2. The immobilized single stranded DNA was then subjected to re-amplification using unlabelled gene specific primer as forward primer as well as for reverse arbitrary priming.

Re-Amplification PCR from the Immobilization Reaction Using Arbitrary PCR

The embodiment of the single primer method involving arbitrary PCR was applied for isolating novel aminoacylase/amidohydrolase genes from two samples (173 and 202B). The same samples were used as in Example 2 and the gene specific primers were also the same as in Example 2. The immobilized single stranded DNA from the first step (linear PCR) was used as a template for the re-amplification. The original degenerate family specific primers AA3 or AA4 (unlabelled) functioned both as a gene specific and an arbitrary primer for retrieval of new aminoacylase/amidohydrolase genes.

The exponential re-amplification PCR was carried out in 50 μl reaction mixture containing 2 μl of the immobilized sample, 1.0 μM unlabelled gene specific primer, AA3 or AA4, (the gene specific primer corresponded to the first linear PCR), 200 μM of each dNTP in 1× DyNAzyme DNA polymerase buffer and 2.0 U DyNAzyme DNA polymerase (Finnzymes, Espoo, Finland) with a MJ Research thermal cycler PTC-0225. The reaction mixture was first carried out by denaturing at 95° C. for 5 min, followed by 30 cycles of denaturing at 95° C. (0:50 min), annealing at 55° C. for 50 s and extension at 72° C. (2 min). This was then followed with a final extension for 7 min at 72° C. to obtain adenine (“A”) overhangs.

Analyzing, Purification and Cloning of the PCR Products

Analysis, purification, and cloning of the PCR products were as described in Example 2. Visible re-amplification DNA products (obtained from pooled samples) of 0.2-0.5 kb were observed on agarose gels for both primers (AA3 and AA4). Inserts in approximately 280 clones were sequenced and 54 (19%) of the cloned sequences gave closest hit to aminoacylase/amidohydrolase sequences, belonging to 11 different aminoacylase/amidohydrolase genes (Table 3 & 7). Amidohydrolase EAA4 was found in sample 173 but the other sequences were found in sample 202B.

### Example 4

### Retrieval of Gene Fragments Coding for Enzymes Belonging to the Glycoside Hydrolase Family 13, Using Single Gene Specific Primer in First Step and Adapter-Supplied Priming Site in Second Step

Samples

Sample Z from Example 1 was used as source DNA.

Construction of Degenerated Primers

For the primer construction, amino acid sequences of various amylolytic enzymes were retrieved from protein sequence databases (Bateman et al., 1999; Maidak et al., 1999) and aligned by using CLUSTALX version 1.8. (Thompson et al., 1994). Furthermore, blocks of multiply aligned amino acid sequences, established with the program Blockmaker (Henikoff et al., 1995) were used as input for the CODEHOP program. Primers were designed according to the CODEHOP strategy by using the CODEHOP program (Rose et al., 1998). The primers were degenerate at the 3′ core region of length 11 bp across four codons of highly conserved amino acids. In contrast, they were non-degenerate at the 5′ region (consensus clamp region) of 13-29 bp with the most probable nucleotide predicted for each position.

Two sequence regions (A and B) separated by ˜80-200 amino acids were chosen as primer target sites for the amylase family 13 (Takehiko, 1995) Subsequently, forward and reverse primers were constructed for family 13, aimed to complement to the DNA coding sequences of the conserved A and B regions, respectively. The primers were Am508 (5′-GATATTTAATATGTTTAGCTGCATCAATTckraanccrtc-3′; degeneracy 32: reverse) (SEQ ID NO: 36); Am510 (5′-GGCGGCGTCGATCckraanccrtc-3′; degeneracy 32: reverse) (SEQ ID NO: 37); Am14 (5′-GATCAACTTAATTAGCAACATCCATTckccanccrtc-3′; degeneracy 16: reverse) (SEQ ID NO: 38) and Am30 (5′-GCCCCGCTGGGTGtcrtgrttntc-3′; degeneracy 16: reverse) (SEQ ID NO: 39) corresponding to region B and primers Am1 (5′-GCATGTTATGCTGGATGCAgtnttyaayca-3′; degeneracy 16: forward) (SEQ ID NO: 40) and Am3 (5′-AAATGTGCAAGTGTATATGGATTTTgtnytnaayca-3′; degeneracy 64: forward) (SEQ ID NO: 41) of region A.

Linear PCR with Single Degenerate Family Specific Primer

The Z sample DNA was used as a template for extending the family 13 amylase gene-specific primers of region B (Am508 and Am510). The primers were biotin labelled at the 5′ end (MWG Biotech, Ebersberg, Germany). The PCR was carried out in 50 μl reaction mixture containing 1-100 ng of genomic DNA (dilutions used), 0.2 μM primer Am508, or Am510, 200 μM of each dNTP in 1× DyNAzyme DNA polymerase buffer and 2.0 U DyNAzyme DNA polymerase (Finnzymes) with a MJ Research thermal cycler PTC-0225. The reaction mixture was first denatured at 95° C. for 5 min, followed by 40 cycles of denaturing at 95° C. (0:50 min), annealing at five different temperatures (40° C., 43.8° C., 50° C., 57.3° C. and 62° C.) for 50 s and extension at 72° C. (2 min). Samples were loaded on 1% TAE agarose to identify unspecific priming. Only those samples giving no visible bands after this first linear PCR (analyzed on agarose gel, as described in Example 2), thus indicating a low unspecific priming, were selected for ligation and re-amplification. They were processed separately by the following protocols.

PCR Purification and Immobilization of Single Stranded PCR Products

Excess of biotin labelled primers, nucleotides and polymerase was removed by passing the PCR samples through QIAquick PCR purification spin columns (QIAGEN, Germany) by following the manufactures instructions. The samples were eluted with 30 μl of dH₂O and then the biotin labelled PCR products were immobilized by using 150 μg of streptavidin-coated magnetic beads (Dynal, Oslo, Norway) according to the instructions of the manufacturer. The captured biotin labelled PCR products were resuspended in 11 μl of dH₂O. PCRs from the different annealing temperatures for each primer of the amylase genes were pooled in the QIAGEN PCR purification step. The immobilized single stranded DNA was then subjected to a ligation reaction as described below.

Ligation of an Adaptor (oli10) to the Single Stranded Biotin Labelled PCR Products Using T4 RNA Ligase

In the presence of 20 U of T4 RNA ligase (New England BioLabs, Beverly, Mass., USA), T4 RNA ligation buffer (50 mM Tris-HCl, pH 7.8, 10 mM MgCl 2, 10 mM DTT and 1 mM ATP) and 10% PEG8000, 50 nM of the adaptor 5′-phosphorylated oligodeoxyribonucleotide oli10 (5′-AAGGGTGCCAACCTCTTCAAGGG-3′; oli10 in FIG. 1A) (SEQ ID NO. 34) was added to the captured DNA in a final volume of 20 μl. The mixture was incubated at 22° C. for 24-60 h.

Re-Amplification PCR from the Ligation Reaction

The exponential reamplification PCR was carried out in 50 μl reaction mixture containing 2 μl ligation mixture, 1.0 μM unlabelled gene specific primer Am508, or Am510, (the gene specific primer corresponded to the first linear PCR), 1.0 μM oli11 (5′-CTTGAAGAGGTTGGCACCCT-3′) (SEQ ID NO. 35) which is complementary to oli10, 200 μM of each dNTP in 1× DyNAzyme DNA polymerase buffer and 2.0 U DyNAzyme DNA polymerase (Finnzymes, Espoo, Finland) with a MJ Research thermal cycler PTC-0225. The reaction mixture was first carried out by denaturing at 95° C. for 5 min, followed by 30 cycles of denaturing at 95° C. (0:50 min), annealing at 55° C. for 50 s and extension at 72° C. (2 min). This was then followed with a final extension for 7 min at 72° C. to obtain ‘A’ overhangs.

Analyzing, Purification and Cloning of the PCR Products

Seven microliters of the PCR products were taken for 1% TAE agarose gel electrophoresis to confirm the identity of the PCR products and the patterns compared between the control PCRs (gene specific primers) and the main PCRs (oli11/gene specific primers). Before cloning, thirty microliters of the PCR products were loaded on thick 1% TAE agarose electrophoresis gels. Bands and smears of approximately 100-2000 bases were excised from the gel and purified by using spin columns, GFX PCR DNA and Gel Band Purification kit according to the manufacturer (Amersham Biosciences, Hørsholm, Denmark). The samples were eluted with 25 μl of dH₂O. Then the purified PCR products (4 μl) were cloned by the TA cloning method (Zhou and Gomez-Sanchez, 2000). Plasmid DNAs from single colonies were isolated and purified by using Multiscreen Separation System according to the instructions of the manufacturer (Millipore Corporation, Bedford, Mass.). The gene inserts were sequenced with M13 reverse and M13 forward primers on ABI 3700 DNA sequencers by using a BigDye terminator cycle sequencing ready reaction kit according to the instructions of the manufacturer (PE Applied Biosystems, Foster City, Calif.). All sequences were analysed in Sequencher 4.0 for Windows (Gene Codes Coperation, Ann Arbor, Mich.) and XBLAST searched (Altschul et al., 1990; Altschul et al., 1997). All sequences were imported into the program BioEdit version 5.0.6 (Tom Hall, North Carolina State University, Department of Microbiology) and aligned there by ClustalW. Approximately 570 clones were sequenced and 45 (8%) of those sequences gave closest hit to amylase sequences, belonging to 10 different amylases (Table 4 & 7).

### Example 5

### Retrieval of Complete Genes from Discovered Fragments

Following the sequencing of the obtained target gene fragments of 4 sequences (am159, am162, am164 and am170), their upstream and downstream flanking regions were amplified from the DNA sample Z in a series of inverse nested PCR reactions in which one primer was specific for the target gene fragment and the other was an arbitrary primer that was targeted to the unknown flanking sequence (Sorensen et al., 1993; Marteinsson et al., 2001a). The gene specific primer was biotin-labelled at the 5′-end and the PCR product was purified using QIAquick PCR purification spin columns prior to a second PCR with a nested gene specific primer upstream to the previous one. The resulting amplification product of the latter PCR reaction was cloned and sequenced. The sequence information was used to make new gene specific primers for subsequent nested PCR amplification. In this manner by series of inverse nested PCR, the complete 5′ and 3′ flanking sequences for genes coding for enzymes am159, am162, am164 and am170 were obtained (Table 5 & 7).

### Example 6

### Retrieval of Gene Fragments Coding for Enzymes Belonging to the Glycoside Hydrolase Family 13, Using Two, Reverse and Forward, Gene Specific Primers

For a comparison with the present invention, PCR screening for glycoside hydrolases of family 13 from sample Z was carried out using two gene specific primers. Four degenerate amylase primers were made from the conserved regions A and B (Am1, Am3, Am14 and Am30 as described above in Example 4). A PCR matrix was prepared by testing both of the forward primers (Am1 and Am3) against both of the reverse primers (Am14 and Am30). The PCR was carried out in 50 μl reaction mixture containing 10-100 ng of genomic DNA, 1.0 μM of both reverse an forward primers (giving 4 different combinations), 200 μM of each dNTP in 1× DyNAzyme DNA polymerase buffer and 2.0 U DyNAzyme DNA polymerase (Finnzymes, Espoo, Finland) with a MJ Research thermal cycler PTC-0225. The reaction mixture was first denatured at 95° C. for 5 min, followed by 30 cycles of denaturing at 95° C. (0:50 min), annealing at 52° C. for 50 s and extension at 72° C. (3 min). This was followed by a final extension for 7 min at 72° C. to obtain ‘A’ overhangs. PCR products were loaded on gels and the resulting bands were excised from the gel and purified by using GFX spin columns as described above. Cloning, plasmid preps, sequencing and sequence analysing were done by using the methodology described above. Approximately 94 clones were sequenced and 13 (14%) of those sequences were identified by homology as amylase sequences, belonging to 4 different amylases, shown in Table 6 & 7.

TABLE 1
Complexity and species plurality of the DNA extracted from
environmental samples Z, 173 and 202B as seen by the frequencies
of OTUs within the Bacteria domain derived from the 16S rRNA
sequences.

| | | |
|---|---|---|
| No. of | | |
| clones | Closest database match | % Match |

Sample Z.

| | | |
|---|---|---|
| 20 | Chloroflexus aurantiacus | 99 |
| 13 | NAK14 | 98 |
| 11 | Thermus NMX2 A.1 | 98-100 |
| 4 | Thermodesulfovibrio sp. | 97 |
| 2 | Meithermus cerbereus | 96 |
| 2 | Uncertain affiliation | <88 |
| 2 | Fervidobacter | |
| | gondwanalandicum | 97 |
| 2 | Chlorogloeopsis sp. | 99 |
| 1 | Calderobacterium | |
| | hydrogenophilum | 97 |
| 1 | Thermocrinis ruber | 94 |
| 1 | Paracraurococcus roseus | 90 |
| 1 | Thiobacillus hydrothermalis | 94 |
| 1 | Thermus ZHGI | 97 |
| 1 | Meiothermus ruber | 99 |
| 62 | Total OTUs | 14 |

Sample 173

| | | |
|---|---|---|
| 34 | Chloroflexus aurantiacus | 99 |
| 30 | Aquificales SRI-240 | 99 |
| | Uncultured gamma proteobacterium BioIuz | |
| 19 | K32 | 99 |
| 18 | Thermus sp. | 99 |
| 6 | Thermus SRI-248 | 98 |
| 4 | Aquificales O1B-6 | 100 |
| 3 | Thermus sp. NMX2 A.1 | 100 |
| 2 | Aquificales O1B-6 | 100 |
| 2 | Bacterium EX-H1 | 87 |
| | Uncultured gamma proteobacterium BioIuz | |
| 2 | K32 | 97 |
| 1 | Uncultured Verrucomicrobia Arctic 95B-10 | 88 |
| | Unidentified green non-sulfur bacterium | |
| 1 | OPB34 | 99 |
| | Uncultured gamma proteobacterium BioIuz | |
| 1 | K32 | 100 |
| 1 | Thermus sp. ZFI A.2 | 99 |
| 1 | Uncultured Thermocrinis sp. clone SUBT-1 | 99 |
| 1 | Thermus sp. NMX2 A.1 | 97 |
| 1 | Thermotogales SRI-251 | 93 |
| 1 | Uncultured bacterium #0649-1N15 | 88 |
| 1 | Thermotogales SRI-25 1 | 97 |
| 1 | Dictyoglomus thermophilum | 94 |
| 1 | Aquificales SRI-240 | 87 |
| 1 | Aquificales O1B-6 | 95 |
| 1 | Thermus NMX2 A.1 | 94 |
| 1 | Thermus O1B-335 | 97 |
| 1 | Thermus ruber | 95 |
| 135 | Total OTUs | 25 |

Sample 202B

| | | |
|---|---|---|
| 7 | Uncultured epsilon proteobacterium 1061 | 98 |
| 5 | Uncultured bacterium from activated sludge | 98 |
| 4 | Uncultured bacterium 5Y6-103 | 97 |
| 2 | Aquificales SRI-240 | 98 |
| 2 | Proteobacterium MBIC3293 | 97 |
| 2 | Hydrogenophaga palleronii | 96 |
| 2 | Herbaspirillurn seropedicae | 96 |
| 2 | Zoogloea sp. (strain DhA-35) | 99 |
| 1 | Unidentified beta proteobacterium | 99 |
| | Uncultured hydrocarbon seep bacterium | |
| 1 | BPC023 | 89 |
| 1 | Uncultured alpha proteobacterium UP1 | 96 |
| 1 | Aeromonas sp. | 99 |
| 1 | Uncultured bacterium 5Y6-105 | 97 |
| 1 | Uncultured bacterium SY6-60 | 93 |
| 1 | Uncultured bacterium #0319-7F1 | 88 |
| 1 | Uncultured marine eubacterium HstpL102 | 93 |
| 1 | Geothrix fermentans | 98 |
| 1 | MTBE-degrading bacterium PM1 | 95 |
| 1 | Aquificales SRI-240 | 99 |
| 1 | Rhodobacter sp. | 98 |
| 1 | Soil bacterium 565D1 | 97 |
| 1 | Uncultured beta proteobacterium SBRH147 | 99 |
| 1 | Agricultural soil bacterium clone SC-I-50 | 96 |
| 1 | Thermus NMX2 A.1 | 99 |
| 1 | Herbaspirillum frisingense | 96 |
| 1 | Uncultured bacterium SY6-75 | 98 |
| 1 | Bacteroides distasonis | 91 |
| 1 | Alpha proteobacterium F0813 | 99 |
| 1 | Rhizosphere soil bacterium clone RSC-II-60 | 94 |
| 1 | Uncultured bacterium 5Y6-60 | 98 |
| 1 | Uncultured bacterium SY6-101 | 97 |
| 42 | Total OTUs | 31 |

TABLE 2
Aminoacylase/amidohydrolase genes retrieved from samples 173 and 202B
with the single primer method (adaptor ligation in the second step). The “% Match”
values refer to sequence identity of the amino acid sequences encoded by the respective
gene fragments, compared to the corresponding amino acid sequences from the found
closest matching database entries. This also applies “% Match” values of Table 3-6

| Gene | No. of | Fragm. | | | | Database |
|---|---|---|---|---|---|---|
| code | clones | length* | Primer | Closest database match | % Match** | accession |
| EAA1 | 1 | 140 | AA3 | Hippurate hydrolase; | 56 | NP_520992 |
| | | | | Ralstonia solanacearum | | |
| EAA2 | 4 | 180 | AA4 | Hippurate hydrolase; | 56 | NP_520992 |
| | | | | Ralstonia solanacearum | | |
| EAA3 | 1 | 270 | AA4 | Hippurate hydrolase, | 55 | NP_533942 |
| | | | | Agrobacterium | | |
| | | | | tumefaciens | | |
| Total | 6 | | | | | |

*Approximate nt length.
**Amino acid sequence identity to nearest database match.

TABLE 3
Aminoacylase/amidohydrolase genes retrieved from samples 173 and 202B
with the single primer method (arbitrary PCR in the second step).

| Gene | No. of | Fragm. | | | | Database |
|---|---|---|---|---|---|---|
| code | clones | length* | Primer | Closest database match | % Match** | accession |
| EAA3 | 1 | 270 | AA4 | Hippurate hydrolase, | 55 | NP_533942 |
| | | | | Agrobacterium | | |
| | | | | tumefaciens | | |
| EAA4 | 12 | 270- | AA3/ | Amino acid | 52 | NP_127000 |
| | | 360 | AA4 | amidohydrolase; | | |
| | | | | Pyrococcus abyssi | | |
| EAA5 | 12 | 300 | AA4 | Hippurate hydrolase; | 62 | NP_520992 |
| | | | | Ralstonia solanacearum | | |
| EAA6 | 6 | 240 | AA4 | Hippurate hydrolase; | 66 | NP_520992 |
| | | | | Ralstonia solanacearurn | | |
| EAA7 | 12 | 300 | AA4 | Hippurate hydrolase; | 63 | NP_520992 |
| | | | | Ralstonia solanacearum | | |
| EAA8 | 1 | 160 | AA4 | Hippurate hydrolase; | 63 | NP_520992 |
| | | | | Ralstonia solanacearum | | |
| EAA9 | 1 | 280 | AA4 | Hippurate hydrolase, | 56 | NP_533942 |
| | | | | Agrobacterium | | |
| | | | | tumefaciens | | |
| EAA1 | 6 | 260 | AA3 | Hippurate hydrolase; | 65 | NP_520992 |
| 0 | | | | Ralstonia solanacearum | | |
| EAA1 | 1 | 250 | AA3 | Hippurate hydrolase; | 60 | NP_520992 |
| 1 | | | | Ralstonia solanacearum | | |
| EAA1 | 1 | 480 | AA3 | Hydrolase; Streptomyces | 43 | T36488 |
| 2 | | | | coelicolor A3(2) | | |
| EAA1 | 1 | 290 | AA3 | Hippurate hydrolase; | 71 | NP_520992 |
| 3 | | | | Ralstonia solanacearum | | |
| Total | 54 | | | | | |

*Approximately nt length.
**Amino acid sequence identity to nearest database match.

TABLE 4
Amylase genes of family 13 retrieved from sample Z with the single primer
method (adaptor ligation in the second step).

| Gene | No. of | Fragm. | | | | Database |
|---|---|---|---|---|---|---|
| code | clones | length* | Primer | Closest database match | % Match** | accession |
| am27 | 1 | 300 | Am508 | Alpha-amylase; | 64 | P29750 |
| | | | | Thermomonospora | | |
| | | | | curvata | | |
| am80 | 1 | 370 | Am508 | Maltodextrin | 43 | NP_308480 |
| | | | | glucosidase; | | |
| | | | | Escherichia coli | | |
| am156 | 1 | 105 | Am510 | 1,4-alpha-glucan | 62 | NP_213496 |
| | | | | branching enzyme; | | |
| | | | | Aquifex aeclicus | | |
| am159 | 2 | 640 | Am508 | Alpha-amylase; | 58 | P20845 |
| | | | | Bacillus megaterium | | |
| am161 | 3 | 410 | Am508 | Alpha-glucosidase; | 24 | Q17058 |
| | | | | honeybee | | |
| am162 | 2 | 500 | Am508 | 4-alpha- | 49 | 086956 |
| | | | | glucanotransferase; | | |
| | | | | Thermotoga | | |
| | | | | neapolitana | | |
| am163 | 2 | 300 | Am508 | Alpha-amylase; | 48 | NP_578206 |
| | | | | Pyrococcus furiosus | | |
| am164 | 14 | 530 | Am508 | 1,4-alpha-glucan | 40 | NP_442003 |
| | | | | branching enzyme; | | |
| | | | | Synechocystis sp. | | |
| am170 | 17 | 570 | Am508 | Alpha-amylase; | 60 | BAA01600 |
| | | | | Pseudomonas sp. | | |
| am173 | 2 | 680 | Am508 | 1,4-alpha-glucan | 76 | NP_484756 |
| | | | | branching enzyme; | | |
| | | | | Nostoc. sp | | |
| Total | 45 | | | | | |

*Approximate nt length.
**Amino acid sequence identity to nearest database match.

TABLE 5
Complete amylase genes retrieved from sample Z.

| Gene | Gene. | | | Database |
|---|---|---|---|---|
| code | length* | Closest database match | % Match** | accession |
| am159-G | 1690 | Alpha-amylase; Bacillus megaterium | 46 | P20845 |
| am162-G | 1360 | 4-alpha-glucanotransferase; | 41 | O86956 |
| | | Thermotoga neapolitana | | |
| am164-G | 2030 | 1,4-alpha-glucan branching enzyme; | 64 | NP_213496 |
| | | Aquifex aeclicus | | |
| am170-G | 1790 | Alpha-amylase; Pseudoalteromonas | 55 | P29957 |
| | | haloplanktis | | |

*Approximate nt length.
**Amino acid sequence identity to nearest database match.

TABLE 6
Amylase genes retrieved from the sample Z with the conventional two primers
method.

| Gene | No. of | Fragm. | | Closest database | | Database |
|---|---|---|---|---|---|---|
| code | clones | length* | Primer set | match | % Match** | accession |
| am80 | 4 | 400 | Am1:Am14 | Maltodextrin | 46 | NP_308480 |
| | | | | glucosidase; | | |
| | | | | Escherichia coli | | |
| am81 | 6 | 470 | Am1:Am30 | Alpha-amylase; | 45 | AAB60935 |
| | | | | Aedes aegypti | | P14898 |
| am82 | 1 | 220 | Am3:Am14 | Alpha-amylase; | 32 | |
| | | | | Dictyoglomus | | |
| | | | | thermophilum | | |
| am103 | 2 | 470 | Am3:Am14 | Amylase like protein; | | |
| | | | and | Drosophila | | |
| | | | Am3:Am30 | melanogaster | 46 | U69607 |
| Total | 13 | | | | | |

*Approximate nt length
**Amino acid sequence identity to nearest database match.

TABLE 7
List of sequences for gene fragments and complete genes retrieved from
environmental DNA in the present invention.

| Sequence ID No | Gene code | Nt length |
|---|---|---|
| 1 | EAA1 | 140 |
| 2 | EAA2 | 180 |
| 3 | EAA3 | 270 |
| 4 | EAA4 | 270-360 |
| 5 | EAA5 | 300 |
| 6 | EAA6 | 240 |
| 7 | EAA7 | 300 |
| 8 | EAA8 | 160 |
| 9 | EAA9 | 280 |
| 10 | am27 | 300 |
| 11 | am80 | 370 |
| 12 | am156 | 105 |
| 13 | am159 | 640 |
| 14 | am161 | 410 |
| 15 | am162 | 500 |
| 16 | am163 | 300 |
| 17 | am164 | 530 |
| 18 | am170 | 570 |
| 19 | am173 | 680 |
| 20 | am159-G | 1690 |
| 21 | am162-G | 1360 |
| 22 | am164-G | 2030 |
| 23 | am170-G | 1790 |
| 24 | am80 | 400 |
| 25 | am81 | 470 |
| 26 | am82 | 220 |
| 27 | am103 | 470 |
| 28 | EAA10 | 260 |
| 29 | EAA11 | 250 |
| 30 | EAA12 | 480 |
| 31 | EAA13 | 290 |

| Sequences | |
|---|---|
| Code: EAA1: | |
| AACCGGGGCATGGGTACCACCGGCGTTGTCGGAATCGTGAAAGCCGGCACG | SEQ ID NO 1 |
| TCGGAGCGCGCCATTGCCCTGCGTGCCGACATGGACGCCTTGCCGACGCAG | |
| GAGTTCAACACTTTTGAGCACGCCAGCCAACACCCTGGAAAG | |
| Code: EAA2: | |
| TGAGTCGTATTACAATTCACTGGCCGTCGTTTACACACCGTGGTTTGGGTA | SEQ ID NO 2 |
| CTACCGGCGTCGTCGGCATCGTGAAGGCAGGCACCTCGGAACGTGCACTGG | |
| CCTTGCGCGCGGATATGGATGCCCTGCCCATGCAAGAGTGCAACAGCTTTG | |
| CCCACACCAGCCAATACCCAGGCAAG | |
| Code: EAA3: | |
| TTACACGAACTCACGGCTTTCCGCCGTGACCTGCATGTTCACCCCGAGCTGG | SEQ ID NO 3 |
| GGTTTGAAGAGGTTTACACTAGCGGGCGGGTCGCAGAGACCCTGCGCCTGT | |
| GCGGTGTGGATGAGGTTCATACGCAGATTGGCAAGACCGGCGTGGTGGCGG | |
| TTATCAAAGGCAAGCGTCAAAGCAGCGGCAAGATGATGGGGCTGCGTGCCG | |
| ACATGGACGCGCTACCGATGGCCGAGCACAACGAGTTCACCTGGAAATCTG | |
| CCAAATCCGGCCTG | |
| Code: EAA4: | |
| CTAAAGCCCGCCCCTCCCCAATGCTACAGCGAAATGGCTCTGTTGTCAAGG | SEQ ID NO 4 |
| AGGCGCAGTATGATACAATTCCCCTTCAGGAGGTGCCGGATGCTCCAAAAA | |
| GCGCAGGAGATTCAAGAACCCCTGGTGGCCTGGCGACGGGAGTTTCACACT | |
| TACCCTGAACTGGGCTTCCGGGAGAGCCGTACAGCCGCCCGGGTGGCCGAA | |
| ATTTTGACCGGACTGGGCTATCGCGTCCGGACGGGCGTTGGGCGGACCGGA | |
| GTGGTGGCGGAGCGGGGGGAGGGGCACCCCATTATTGCCGTGCGCGCCGAT | |
| ATGGATGCCCTGCCGATCCAGGAGGCCAACGACGTCCCCTATGCCTCTCAG | |
| CACCC | |
| Code: EAA5: | |
| CTGCCTGAACTGCTGGACCAGGCCGATGCCATGCGGGCTTTGCGGCGCGAC | SEQ ID NO 5 |
| ATCCATGCGCACCCCGAGCTGTGTTTTCAAGAAGTACGCACCTCAGACCTGA | |
| TCGCCAAGACCTTGCAAAGCTGGGGCATTGAGGTGCACACGGGTCTGGGCA | |
| CGACCGGTGTCGTGGGCGTGATCAAAGGGCGCCCCGGCAAGCGGGCCATTG | |
| GCTTGAGGGCAGACATCGACGCCCTGCCCATGACCGAGCACAACACCTTG | |
| CCCATGCCAGCCGACACGCGTGTAAAACGACGGCCCAGGGAA | |
| Code: EAA6: | |
| GGTGACGCGCTCACCGAACGAGTGGGTGAGTTCATACAGCTCAGGCGTGAC | SEQ ID NO 6 |
| ATTCATCGCCACCCCGAGCTGGCGTTTGAAGAGCATAGAACGTCCGAGCTG | |
| GTCGCTGCCAAGCTGGAGAGCTGGGGCTACGCGGTGCGTCGCGGCCTGGGT | |
| GGAACCGGAGTGGTGGGTGTTTTAAAGCGCGGCCACAGTCAACGCAGTCTG | |
| GGCATTCGTGCCGACATGGACGCGCTGCCCATTCAGGAGG | |
| Code: EAA7: | |
| CCTTCGTTGCCACCTTCCGTCCTGCCTGAACTGCTGGACCAGGCCGATGCCA | SEQ ID NO 7 |
| TGCGGGCTTTGCGGCGCGACATCCATGCGCACCCCGAGCTGTGTTTTCAAGA | |
| AGTACGCACCTCAGACCTGATCGCCAAGACCTTGCAAAGCTGGGGCATTGA | |
| GGTGCACACGGGTCTGGGCACGACCGGTGTCGTGGGCGTGATCAAAGGGCG | |
| CCCCGGCAAGCGGGCCATTGGCTTGAGGGCAGACATCGACGCCCTGCCCAT | |
| GACCGAGCACAACACCTTTGCCCATGCCAGCCGACACGCGGGCCGCAT | |
| Code: EAA8: | |
| GGCATTCCCCTCCACCGTGGCATGGGCACCACCGGTGTCGTCGGTATCGTCA | SEQ ID NO 8 |
| AAAGCGGGACATCTGATCGGGCTATTGGATTGCGCGCTGACATGGATGCGC | |
| TGCCTATGGCTGAAGCCAACACCTTTGCGCACGCCAGCACCCACCCAGGCA | |
| AGA | |
| Code: EAA9: | |
| ATTACCGAGTTTCATCCCGAACTCACGGCTTTCCGGCGTGACCTGCATGTTC | SEQ ID NO 9 |
| ACCCCGAGTTGGGGTTTGAAGAGGTCTACACCAGCGGGCGGGTTGCTGAGG | |
| GCTTGCGCCTGTGCGGCGTGGATGAGGTCCATACGCAAATTGGCAAGACCG | |
| GCGTGGTGGCTGTTATCAAAGGCAAGCGTCAAACCAGCGGCAAGATGATAG | |
| GGCTGCGTGCCGACATGGACGCGCTACCAATGGCCGAGCACAACGAGTTCA | |
| CCTGGAAATCTGCCAAGACC | |
| Code: am27: | |
| ATGGTTGCCCGTTGCAAAGCGGTCGGTGTTGACATTTATGTTGATGCGGTCA | SEQ ID NO 10 |
| TCAATCATATGACCGGCGTCGGCAGCGGTGTCGGATCGGCTGGCTCAACGT | |
| ATAGCCCGTACAACTATCCGGGCATCTATCAATATCAGGATTTTCACCACTG | |
| CGGCAGAAATGGCAACGATGACATCCAGAATTATGGTGATCGGTACGAAGT | |
| TCAGAACTGCGAACTGGTGAATCTTGCCGATCTCGATACCGGATCATCGTAT | |
| GTGCGGGATCGCTTAGCTGCCTATTTGAACGATCTCATCA | |
| Code: am80: | |
| ATATGTTTAGCTGCATCAATTCGGAAACCGTCAAACCACAAATACGATGTC | SEQ ID NO 11 |
| GAAGACTATACCAGCATTGACCCTCACCTGGGAGGTGAAGCAGGGTTACTC | |
| CTCTTACGCGAGGTACTCGACGAGCGAGCCATGAAGCTGGTGCHGACATC | |
| GTCCCGAACCTTGTGGAGTGACCCATCCGTGGTTTGTCGCTGCCCAGGCCA | |
| ACCCACGATCACCAACAGCCGAGTTCTTCATGTTCCGTCGTCATCCCGACGA | |
| CTACGAGAGCTGGCTGGGGGTCAAGACCCTGCCCAAACTCAATTACCGCAG | |
| TGTCCGCCTCCGCGACGTAATGTACGCAGGCCAGGATGCGATTATGCGCTA | |
| CTGGTTGCGACCAC | |
| Code: am156: | |
| CGCAAACCGGAAGAGGATAACCGTCCGCTCAATTACCGTGAACTGGCCCAC | SEQ ID NO 12 |
| GAGCTGGCCGAGCATGNGAAAGATTGTGGCTTTACCCACGTTGAGCTGTTA | |
| CCG | |
| Code: am159: | |
| ACGGCTGCTACATCCACTCCCACCCTCACAATCACTCCGACCACTAGTCCAA | SEQ ID NO 13 |
| TAGATAAACCGGAATGGTGGAAATCGGCGGTTTTCTATCAGGTGTTTGTGCG | |
| CANTTTTTATGACTCTGATGGAGATGGAATTGGCGATTTTCAGGGATTGATT | |
| CAGAAGCTGGACTATTTGAATGATGGTGATCCCAAAACGAACAGTGATTTG | |
| GGGATTAATGCCGTTTGGTTGATGCCTGTTAATCCCTCGCCGTCTTATCACG | |
| GGTACGATGTGACCGATTACTACAATGTGAATCCCGATTACGGAACGATGG | |
| ATGATTTCAGGGAATTGATAAAGGAGGCTCATCAGCGCGGCATTAAAGTAA | |
| TTATTGATTTGGTGATCAATCATACATCTACTCAGCACCCCTGGTTTCAACA | |
| GGCATTAGACCCCCAATCTCCTTACCATAATTATTACATCTGGCGGGACGAA | |
| AATCCGGGTTACAGCGGACCGGATGGACAAAAGGTCTGGCATCGCGCCTCG | |
| AATGGGAAATATTACTACGCGCTTTTCTGGGATCAAATGCCTGACCTGAACT | |
| TCCAGAATCCGCAGGTCACTGAGGAAATTTATCAGATCGCTCGTTTCTGGCT | |
| GGAAGATGTGGGTGTGGACG | |
| Code: am161: | |
| TACAACGACAACATATCCACCGCCGGACCGTTCAACTTCCTGCCTTCGCCCCG | SEQ ID NO 14 |
| CGCTCAAAGTGACGCTGGTTGGTCTGGGGTATCGGCTCAACAATCAGACTTT | |
| CTATCCCGACTATCAGAGTGAGGTGATGGGTGCCGTCTCACTGGTGCGGCG | |
| AATGTTCCCCCTGGCCAACTCAGCCGGTGGATCAGGTCTCGCCTGGGATTAC | |
| TGGCACATCATGGATGAAGGACTCGGCTCGCGTGTGAACATGACCAATGTC | |
| GAGTGTAACGATTATATCTCGTGGGAAGACGGCAAGGTGGTGGATCGGCGT | |
| AACCTGTGTTCGACCCGCTACGCTAATCACCTGCTCGCCTATCTGCGATCGG | |
| CATGGAAATACAGCGACCGCCTGTEGCCTACGGCCTGATTTCTACCAAT | |
| Code: am162: | |
| ATGATAGGTTACGAGATATTTGTGAGGTCTTTGCGGACTCAAATGATGACG | SEQ ID NO 15 |
| GAATTGGGGATTTCAAAGGCATCGCCCAGAAAGTCGACTATTTCAAGATGC | |
| TCGGCGTAGACTTAATCTGGTTAACGCCGCACTTCAAGTCACCAAGTTACCA | |
| CGGTTACGACATAATCGACTACTTTGACACGAATGTCTCGTTCGGAACACTT | |
| GCAGATTTTAGAGATATGGTCGACAAGCTGCATGCGAATGGAATAAAAATT | |
| GTCATCGACCTGCCGTTCAACCACGTCTCAGACAGGCACCCATGGTTCAAA | |
| GCCGCTATGAACGGCGAAAAACCGTATGTTGATTACTTCCTCTGGGCGCAG | |
| CCGCACTTCAATTTGAAAGAAAAAAGACACTGGGACGAAGAATTGCTTTGG | |
| CACACGAGAAATGGCAAGACATACTACGGCGTGTTCGGTGGTTCTTCGCCC | |
| GACTTGAATTATGAAAACCCCGAAGTTGTGCAAAAT | |
| Code: am163: | |
| CGTGAGACGCCGATTCTTCAGTGGTTCCAGACCGATTACCGCACCATTTTGC | SEQ ID NO 16 |
| AGCGTCTGCCTGAAGTAGTGCAGGCGGGCTACGGCGCGATTTACCTCCCCTC | |
| GCCCGTCAAGTCTGGCGGTGGGGGGTTCAGCACGGGCTACAACCCCTTCGA | |
| TCTGTTTGACTTGGGCGACCGCTTCCAGAAAGGCACTGTACGAACGCAATA | |
| CGGCACGACTCAGGAACTGATAGAGCTGATTCGCCTTGCGCAGCGACTGGG | |
| GCTGGAGGTCTATTGCGACTTGGTGACCAACCATGCGGACAA | |
| Code: am164: | |
| ATGAGTGATACCGAAAAACCTCGCCGCACCCGCCGTAAACAGGTGGCGAAT | SEQ ID NO 17 |
| ACTGATGAGCCTTCCACGACAGTGACGGCCTCGACCACGGATGCACCAACC | |
| GCAACCATTGAGGAACCTFFCGGCGGCTGCTCGTGCTATGATGACCAGTATCC | |
| TCAGCGAGGATGATATTTATCTGTTCAACCAGGGCACCCATTACCGCTTGTA | |
| CGACAAATTTGGTGCTCAGCCGGTGGTGCTGGAAGGTGTACCGGGCACCTA | |
| TTTTGCGGTTTGGGCACCAAATGCCGAGTATGTGGCCGTGATCGGCGACTGG | |
| AATAACTGGGACGCCGGTGCCAACCCGCTCCGGCAGCGCGGCTTTTCGGGT | |
| GTGTGGGAGGGATTTATCCCCCACGTCGGTAAAGGCATGCGCTACAAGTTC | |
| CACATCGCCTCGCGCTACTACGGCTATCGCGAAGACAAGACAGATCCCTTC | |
| GGCACCTACTTCGAGGTCGCACCGCAGACGGCTGCCATTATCTGGGATCGC | |
| GATTACACCTGGTCGGA | |
| Code: am170: | |
| AGTAGTCTTCCGTTCGGTCCGGTGCACCATTCAACCGCACGTGCCCAAACCT | SEQ ID NO 18 |
| CATCACCACGTACCGTATTTGTTCATCTCTTTGAATGGAAGTGGACGGACAT | |
| TGCCCAGGAATGCGAGAACTTTCTGGGGCCACGCGGCTTTGCGGCAGTGCA | |
| GGTGTCGCCACCGCAAGAGCACGCGATTGTTGCCGGTTATCCGTGGTGGCA | |
| ACGGTATCAACCGGTCAGTTATCAATTGACCAGTCGTAGCGGGACACGGGC | |
| TGAATTCGCCAATATGGTTGCCGTTGCAAAGCGGTCGGTGTTGACATTTAT | |
| GTTGATGCGGTCATCAATCATATGACCGGCGTCGGCAGCGGTGTCGGATCG | |
| GCTGGCTCAACGTATAGCCCGTACAACTATCCGGGCATCTATCAATATCAGG | |
| ATTTTCACCACTGCGGCAGAAATGGCAACGATGACATCCAGAATTATGGTG | |
| ATCGGTACGAAGTTCAGAACTGCGAACTGGTGAATCTTGCCGATCTCGATA | |
| CCGGATCATCGTATGTGCGGGATCGCTTAGCTGCCTATTTGAACGATCTCAT | |
| CATG | |
| Code: am173: | |
| CTGTTTCCAGAAAAACTGGGAGCGCACCCCACAGAAATAGACGGCGTTAAG | SEQ ID NO 19 |
| GGTGTTTATTTTGCCGTTTGGGCTCCCAATGCACGTAACGTTTCCGTGATTG | |
| GCGATTTCAATCAGTGGGATGGACGCAAACATCAGATGCGTAAAGGACAAA | |
| CTGGGGTTTGGGAATTGTTTATTCCTGAACTTGGGGTAGGAGAACATTACAA | |
| ATACGAAATCAAAAATCTAGAAGGTCACATTTACGAAAAATCTGACCCCTA | |
| CGGTTTCCAACAAGAACCTCGTCCCAAAACAGCATCGATTGTCACTGACTTA | |
| AATAGCTATCAGTGGAACGACGAAGATTGGATGGAGCAGCGGCGTCACACC | |
| TATCCTCTGACTCAACCCATCTCAGTTTACGAAGTACATTTAGGTTCTTGGTT | |
| ACACGCCTCTAGCGCAGAACCACCTAGACTACCTAATGGGGAAACCGAGCC | |
| TGTCGTTCCTGTTTCTGAACTTAATCCTGGTGCGCGTTTTCTGACTTATCGAG | |
| AGCTAGCAGACAGGTTAATCCCCTACGTCAAAGATTTGGGCTATACCCATGT | |
| GGAATTATTGCCTATCGCTGAACATCCCTTTGATGGTTCTTGGGGTTACCAA | |
| GTCACAGGCTATTACGCCCCTACTTCCCGTTATGGTAGCCCAGAAGATTTTA | |
| TGTATTTTGTTG | |
| Code: am159-G: | |
| GTGACCTGGTACGAGGGCGCTTTCTTCTACCAGATCTTTCCCGACCGCTACT | SEQ ID NO 20 |
| TCCGGGCTGGCCCTTTCGGAAAGCCAGTCCCGGTAGGGGCTTTGGAACCCT | |
| GGGAAACACCCCCCATCCCTTAGGGGCTKCAAGGGCGGGACCCTCTGGGGCA | |
| TAGCGGAGAAAATCCCCTACCTCAAGGACCTGGGGGTGGAAGCCCTTTACC | |
| TGAACCCCGTCTTCGCCTCCACCGCCAACCACCGGTACCACACCACGGACTA | |
| TTTCCAGGTGGATCCCCTCCTGGGGGGGAACGTGGCCCTAAGGCACCTCCTG | |
| GAAGTCGCCCACGCCCACGGCATGCGGGTCATCCTGGACGGGGTCTTCAAC | |
| CACACGGGTAGGGGCTTTTTTGCCTTCCAGCACCTTCTGGAAAACGGAGAA | |
| CAAAGCCCCTACCGGGACTGGTACCACGTGAAGGGTTTTCCCCTAAACCCCT | |
| ATAGCCGCCACCCCAACTACGAGGCCTGGTGGGGCAATCCTGAGCTTCCCA | |
| ARCTCCGGGTGGAAACCCCGGCGGTGCGGGAGTACCTCCTGGAGGTGGCGG | |
| AGCACTGGATCCGCTTCGGCGCGGATGGCTGGCGGCTGGACGTGCCCAACG | |
| AGATCCCCGACCCCGAGTTCTGGCGGGCCTTCCGCAGGAGGGTGAAGGGGG | |
| CGAACCCGGAGGCCTACCTCGTGGGGGAGATCTGGGAGGAGGCCGAGGCCT | |
| GGCTCCAGGGGGACATCTTTGACGGGGTGATGAACTACCCCCTCGCCCGGG | |
| CGGTTCTAGGCTTCGTGGGAGGGGAGGCCCTGGACCGGGAGCTTGCCGCCC | |
| GCTCGGGCCTAGGGCGGGTGGAACCCCTCCAGGCCCTGGCCTTCAGCCACC | |
| GCCTCGAGGACCTTTTCGGCCGGTATCCCTGGGCGGCGGTCCTGGCCCAGAT | |
| GAACCTCCTCACCTCCCACGACACCCCGAGGCTCCTCTCCCTCCTCCGGGGG | |
| GACGTGGCCCGGGCGCGCCTGGCCCTGAGCCTCCTCTTCCTCCTCCCGGGAA | |
| ACCCCACGGTCTACTACGGGGAGGAAGTGGGGATGGAGGGCGGCCCTGACC | |
| CCGAGAACCGCGGGGGGATGGTGTGGGAGGAAGGGCGCTGGCGGGGGGAG | |
| CTCCGCGAGGCGGTGAGGAGGATGGCGAGGCTGCGCCAGGCCCATCCCGAG | |
| CTCCGCACCGCCCCCTACCGGCGGGTCTACGCCCAGGACCGGCACCTGGCC | |
| TTCACCCGCGGGCCCTACCTGGCGGTGGTGAACGCCAGCGACCGCCCCTTCC | |
| GGCAGGACCTTCCCCTGCACGGCGTCTTCCCCCGGGGGGGTGAGGCCCTGG | |
| ACCTCCTCTCGGGGGCCCGGGCCAAGCTCCAGGGGGGAAGGCTCCTGGGCC | |
| CCGAGCTGCCCCCCTTCGCCCTCGCCCTGTGGCAGGAGGTGTGA | |
| Code: am162-G: | |
| ATGATAGGTTACGAGATATTTGTGAGGTCCTTTGCGGACTCAAATGATGACG | SEQ ID NO 21 |
| GAATTGGGGATTTCAAAGGCATCGCCCAGAAAGTCGACTATTTCAAGATGC | |
| TCGGCGTAGACTTAATCTGGTTAACGCCGCACTTCAAGTCACCAAGTTACCA | |
| CGGTTACGACATAATCGACTACTTTGACACGAATGTCTCGTTCGGAACACTT | |
| GCAGATTTTAGAGATATGGTCGACAAGCTACATGCGAATGGAATAAAAATT | |
| GTCATCGACCTGCCGTTCAACCACGTCTCAGACAGGCACCCATGGTTCAAA | |
| GCCGCTATGAACGGCGAAAAACCGTATGTTGATTACTTCCTCTGGGCGCAG | |
| CCGCACTTCAATTTGAAAGAAAAAAGACACTGGGACGAAGAATTGCTTTGG | |
| CACACGAGAAATGGCAAGACATACTACGGCGTGTTCGGTGGTTCTTCGCCC | |
| GACTTGAATTATGAAAACCCCGAAGTTGTGCAAAAATCACTCGAGATAGTT | |
| GAATTCTGGCTCAAGCAGGGCGTTGATGGATTCAGATTTGATGCGGCAAAG | |
| CACATATACGACTACGATATCAAAGAAGGCAAATTCAGATACGACCACGAA | |
| AAGAATGTCGCCTATTGGCAACTCGTTATGGACAGAGCAAGGCAAATCAAA | |
| GGAGAAGATGTATTCGCAGTTACGGAAGTCTGGGACGATCCTGAAATCGTT | |
| GACAGGTACGCTAAGACAATCGGCTGTTCGTTCAACTTCTACTTCACAGAAG | |
| CCATAAGAGAATCGATGCAGCACGGAGCGGTGTACAAAATCGTCGACTGCT | |
| TTCAGAGAACACTCACGAAAAAGCCATACCTGCCAAGCAACTTCACAGGCA | |
| ACCACGACATGCACAGACTGGCTCAGCTACTACCACATGAAGAGCAGAGAA | |
| AAGTCTTCTTCGGACTGCTCATGACAACACCCGGCGTTCCGTTCATATACTA | |
| CGGCGATGAGCTCGGAATGAAGGGGCAGTACGACTCCACATTCACAGAAGA | |
| CGTTATAGAACCATTCCCATGGTACGCTTCGCTATCTGGCGAGGGCCAAGCG | |
| TTCTGGAAGGCTGTAAGGTTCAACAGGGCATTCACCGGTGCTTCTGTTGAGG | |
| AACACCTGAACCGCGAGGACAGTCTGCTCAAAGAAGTTATTAACTGGACAA | |
| AGTTCAGGAAAACGACTGGCTCACAAACGCATGGGTAGAGCACGTA | |
| ACGCACAACACGTTCACAATCGCTTATACGGTTACAGACGGCGACAACGGA | |
| TTCAGAGTTTATGTGAACATAGCTGGCCACCACGAGACCTTCGAAGGAGTA | |
| AGTCTCAAAGCGTACGTTAAGGTTCTCTGA | |
| Code: am164-G: | |
| ATGAGTGATACCGAAAAACCTCGCCGCACCCGCCGTAAACAGGTGGCGAAT | SEQ ID NO 22 |
| ACTGATGAGCCTTCCACGACAGTGACGGCCTCGACCACGGATGCACCAACC | |
| GCAACCATTGAGGAACCTTCGGCGGCTGCTCGTGCTATGATGACCAGTATCC | |
| TCAGCGAGGATGATATTTATCTGTTCAACCAGGGCACCCATTACCGCTTGTA | |
| CGACAAATTTGGTGCTCAGCCGGTGGTGCTGGAAGGTGTACCGGGCACCTA | |
| TTTTGCGGTTTGGGCACCAAATGCCGAGTATGTGGCCGTGATCGGCGACTGG | |
| AATAACTGGGACGCCGGTGCCAACCCGCTCCGGCAGCGCGGCTTTTCGGGT | |
| GTGTGGGAGGGATTTATCCCCCACGTCGGTAAAGGCATGCGCTACAAGTTC | |
| CACATCGCCTCGCGCTACTACGGCTATCGCGAAGACAAGACAGATCCCTTC | |
| GGCACCTACTTCGAGGTCGCACCGCAGACGGCTGCCATTATCTGGGATCGC | |
| GATTACACCTGGTCGGATCAACAGTGGATGAGCGAACGGGGGCAGCGGCA | |
| GCGCCTCGATGCGCCGATCTCCATCTACGAAGTGCATTTGGGATCGTGGCGG | |
| CGCAAACCGGAAGAGGATAACCGTCCGCTCAATTACCGTGAACTGGCCCAC | |
| GAGCTGGTCGAGCATGTGAAAGATTGTGGCTTTACCCACGTTGAGCTGTTAC | |
| CGGTCACCGAGCATCCCTTCTACGGTTCCTGGGGGTATCAATCGACGGGTTT | |
| GTTCGCGCCGACCAGCCGGTACGGAACGCCGCAAGACTTCATGTATTTTGTG | |
| GATTATCTGCATCAAAACGGGATTGGGGTGATCCTCGATTGGGTGCCCAGC | |
| CACTTCCCGACCGACGGTCATGGGCTGGCCTACTTCGATGGTACCCATCTCT | |
| ACGAACACGCCGATCCGCGTAAAGGCTACCATCCCGACTGGGGAAGCTATA | |
| TTTACAACTATGGTCGGAACGAGGTACGAAGCTTCCTGATCASGCTCGGCGCT | |
| CTGCTGGCTGGATAAGTTTCACATTGACGGGATACGGGTTGATGCGGTTGCG | |
| AGCATGCTCTATCTCGACTATTCGCGCCGAGCCGGCGAGTGGATTCCCAACG | |
| AATACGGTGGGAACGAAAATCTGGAGGCGATTAGCTTCCTGCGCGAATTGA | |
| ACACCCAGATTTACAAGTACTACCCTGATGTGCAGACAATTGCCGAGGAGA | |
| GCACAGCCTGGCCGATGGTATCGCGACCGGTCTACGTTGGTGGATTGGGCTT | |
| CGGCTTCAAGTGGGACATGGGCTGGATGCACGATACCCTGCAGTATTTCCG | |
| GCGCGATCCGATCTACCGGCGCTTTCATCACAACGAATTGACCTTCCGTGGC | |
| CTCTACATUITCAGCGAGAACTACGTGCTACCACTCTCGCACGATGAGGTCG | |
| TTCACGGCAAAGGGTCACTGCTCGACAAGATGGCCGGCGATGTCTGGCAAA | |
| AGTTTGCCAACCTGCGCCTGCTCTACAGCTATATGTTTGCTCAACCCGGTAA | |
| AAAACTGCTCTTCATGGGTGGTGAATTCGGACAGTGGCGCGAATGGTCACA | |
| CGACACCAGCCTGGACTGGCACTTACTGATGTTCCCTCCCATCAGGGCGTA | |
| CAACGATTGNTTGGCGATCTTAACCGTCTCTACCGTACTGAGCCGGCCTTGC | |
| ACGAACTGGACTGTGATCCACGTGGGTTTGAGTGGATCGATGCCAATGATG | |
| CCGATGCCAGCGTCTACAGCTTTCTGCGCAAGAGCCGCTACGGCGAGCAAA | |
| TTCTGATCGTGATCAATGCCACGCCGGTCGTGCGTGAGGATTACCGAATTGG | |
| GGTACCGGTGGGTGGCTGGTGGCGTGAATTGTTTAACAGCGACTCGGAGTA | |
| TTATTGGGGAAGTGGGCAAGGCAATGCCGGCGGCGTGATGGCCGAAGCAAT | |
| TCCAACCCATGGCCGGGATTTTTCGTTGCGACTGCGCCTGCCGCCCCTGGGT | |
| GCGCTCTTCCTGAAACCTGCCGGCTAA | |
| Code: am170-G: | |
| TCATTCCACTACTCACTGTTGTTGAGTCTGGTCAGCGTTGGCCGCTTCCTGG | SEQ ID NO 23 |
| AGCAAAGGAGCCTGTTTATGCCCGGCACTCGCTTTCCCTCGCTTCGTCGGCT | |
| CGTCCTCGTTGTCGCCCTTCTCATGGTGGTAAGTAGTCTTCCGTTCGGTCCGG | |
| TGCACCATTCAACCGCACGTGCCCAAACCTCATCACCACGTACCGTATTTGT | |
| TCATCTCTTTGAATGGAAGTGGACGGACATTGCCCAGGAATGCGAGAACTT | |
| TCTGGGGCCACGCGGCTTTGCGGCAGTGCAGGTGTCGCCACCGCAAGAGCA | |
| CGCGATTGTTGCCGGTTATCCGTGGTGGCAACGGTATCAACCGGTCAGTTAT | |
| CAATTGACCAGTCGTAGCGGGACACGGGCTGAAWTCCCCCATATGGTTGCC | |
| CGTTGCAAAGCGGTCGGTGTTGACATTTATGTTGATGCGGTCATCAATCATA | |
| TGACCGGCGTCGGCAGCGGTGTCGGATCGGCTGGCTCAACGTATAGCCCGT | |
| ACAACTATCCGGGCATCTATCAATATCAGGATTTTCACCACTGCGGCAGAA | |
| ATGGCAACGATGACATCCAGAATTATGGTGATCGGTACGAAGTTCAGAACT | |
| GCGAACTGGTGAATCTTGCCGATCTCGATACCGGATCATCGTATGTGCGGG | |
| ATCGCTTAGCTGCCTATTTGAACGATCTCATCAGTCTGGGAGTTGCCGGTTT | |
| TCGGATTGACGCAGCTAAACACATTGCTGCCGGGGATATTGCCGCAATTTTA | |
| TCCCGTGTGAATGGGAGTCCGTACATTTACCAGGAAGTGATCGGTGCGGCT | |
| GGCGAACCGATTACACCGTGGGAATACACAAATAATGGTGATGTCACTGAA | |
| TTTAAGTATAGCAACGAGATCGGGCGGGTCTTTTTGAATGGTAAGCTGGCAT | |
| GGCTGAGTCAGThGGCGAAGCCTGGGGGATGCTGCCAAGCGACAAAGCGA | |
| TTGTCYFCGTTGATAATCACGACAACCAGCGCGGGCATGGCGGTGGTGGGA | |
| CTGTGGTCACATACAAGAATGGTGTGCTGTACGATCTGGCAAACGTGTTTAT | |
| GCTAGCGTGGCCGTATGGGTACCCCCAGGTGATGTCAAGTTATGAGTTTAGC | |
| AATGATTTTCAAGGGCCACCGAGTGATGCGAACGGCAACACGCGCAGCGTC | |
| TATGTTAACGGNCAGCCCAATTGCTTTGGCGAATGGAAATGCGAGCATCGC | |
| TGGCGACCAATTGCGAATATGGTAGCGTTCCGCAATGCCACAGCGAGTACA | |
| TTCAGTGTGAGTGATTGGTGGAGTAACGGCAACAACCAGATCGCCTTTGGT | |
| CGTGGCGATAAAGGGTTTGTCGTTATCAATCGTGAGGATACAACGCTGAAT | |
| CGCACGTTTCAGACGAGTATGGCGCCTGGGGTCTACTGCAATGTGATTGTTG | |
| CCGTTTTACAAACGGTACGTGCAGTGGGCAAACCGTCACCGTGGACAGTA | |
| ATCGACGGATAACGGTCTCTATTCCGCCTTTCAGTGCTCTTGCCATCCATGT | |
| AGGAGCGAAGTTGTCTACGCAACCGGCAACTGTTGCGGTTTACTTTCAACGT | |
| GAATGCGACGACCTACTGGGGGCAGAACGTGTTTGTGGTTGGGAATATCCC | |
| GCAATTGGGCAACTGGAACCCGGCGCAGGCTGTGCCCCTTTCAGCGGCTAC | |
| GTATCCGGTCTGGAGTGGTACCGTTAATCTGCCGGCAAATACCACCATCGA | |
| ATACAAGTACATTAAGCGTGACGGATCAAATGTGGTGTGGGAGTGTTGTAA | |
| TAATCGCGTTATTACGACGCCAGGTAGTGGCTCGATGACGCTGAATGAGAC | |
| GTGGCGTCCGTGA | |
| Code: am80: | |
| ACCGATCTGGGAGTCTCGGCACTGTACCTCAATCCTATCTTCCGAGCGCCGT | SEQ ID NO 24 |
| CGAACCACAAATACGATGTCGAAGACTATACCAGCATTGACCCTCACCTGG | |
| GAGGTGAAGCAGGGTACTCCTCTTACGCGAGGTACTCGACGAGCGAGCCA | |
| TGAAGCTGGTGCTTGACATCGTCCCGAACCATTGTGGAGTGACCCACCCGTG | |
| GTTTGTCGCTGCCCAGGCCAACCCACGATCACCAACAGCCGAGTTCTTCATG | |
| TTCCGTCGTCATCCCGACGGCTACGAGAGCTGGCTGGGGGTCAAGACCCTG | |
| CCCAAACTCAATTACCGCAGTGTCCGCCTCCGCGACGTAATGTACGCAGGC | |
| CAGGATGCGATTATGCGCTACTGGTTGCGACCACCCTATCGGATC | |
| Code: am81: | |
| GCCGTTGTTTGATTAGCGATTACAGTGATCGCTATCAGGTCCAGTATTGTC | SEQ ID NO 25 |
| AGTTAGCCGGCCTGCCAGACCTCGATACCGGTAAGAGCACTGTGCAGACGA | |
| AGCTGCGTGCTTACCTGCAAGCCCTGCTCAATGCCGGTGTCAAAGGCTCCG | |
| CATTGATGCTGCCAAGCACATGGCCGCGCACGAGGTCGGTGCCATTCTCGA | |
| TGGGCTGACCCTCCCCGGCGGCGGTCGTCCGTACATCTTCAGTGAAGTCATT | |
| GACATGGATCCCAATGAGCGGATACGCGATTGGGAATACACGCCTTACGGA | |
| GACGTCACCGAGTTTGCCTACAGTATTAGCGTGATCGGGAATACCTTCAATT | |
| GTGGTGGATCGCTCAGCAATCTGCAAAACTTCACCACGAACCTACTGCCCTC | |
| GCACTTCGCCCAGATTTTCGTTGACAACCACGACACCCAGCGGGGCAAGGG | |
| CGAATTCGTT | |
| Code: am82: | |
| GGCGAGATTGTTGATCCCTCCGATGTTCAAATGGCCTTTGCCGGGCAACTGG | SEQ ID NO 26 |
| ATGGCGCGCTAGACTTTATCTTGCTGGAAGGTTTGCGTCAGGCTATCGCCATT | |
| TGGGCGCTGGAATGGCTTTCAACTTGCCTCGTTTTTAGAACGGCACCAGATT | |
| TATTTTCCGGAAGTTTCTCTCGTCCATCGTTCTTGGACAACCACGACACCC | |
| AGCGGGGCAAGGGC | |
| Code: am103: | |
| GATTTTCACGCCGATTGTTTGATTAGCGATTACAGTGATCGCTATCAGGTCC | SEQ ID NO 27 |
| AGTATTGTCAGTTAGCCGGCCTGCCAGACCTCGATACCGGTAAGAGCACTG | |
| TGCAGACGAAGCTGCGTGCTTACCTGCAAGCCCTGCTCAATGCCGGTGTCA | |
| AAGGCTTCCGCATTGATGCTGCCAAGCACATGGCCGCGCACGAGGTCGGTG | |
| CCATTCTCGATGGGCTGACCCTCCCCGGCGGCGGTCGTCCGTACATCTTCAG | |
| TGAAGTCATTGACATGGATCCCAATGAGCGGATACGCGATTGGGAATACAC | |
| GCCTTACGGAGACGTCACCGAGTTTGCCTACAGTATTAGCGTGATCGGGAA | |
| TACCTTCAATTGTGGTGGATCGCTCAGCAATCTGCAAAACTTCACCACGAAC | |
| CTACTGCCCTCGCACTTCGCCCAGATTTTCGTTGACAACCACGACACCCAGC | |
| GGGGCAAGGGC | |
| Code: EAA10: | |
| ATGAAACTGATAGACAGCATTGTGCAAAACACACCGACGATCGCGGCGGTG | SEQ ID NO 28 |
| CGACGCGATCTGCACGCCCACCCCGAATTGTGTTTTGAGGAAAACCGCACG | |
| GCCGACAAGGTCGCATCCAAGCTCGCGGAGTGGGGCATCCCGTTCCATCGT | |
| GGCCTTGCGACTACTGGCGTGGTGGGCATCATCCAGTCGGGCACTTCTGACA | |
| GAGCCATTGGCTTGCGCGCTGATATGGACGCGTTGCCGATGCAAGAGGTCA | |
| ATACCTT | |
| Code: EAA11: | |
| ATGAACCTTATTGACTCCATTGTTTCCAGCGCCGCGTCCATTGCAGCCGTCC | SEQ ID NO 29 |
| GCCGCGATCTACATGCCCCATCCGGAGCTGTGTTTTAAGGAAGTGCACACTTC | |
| CGATGTCGTGGCACAGCGGCTGACCGATTGGGGTATCCCGATTCACCGCGG | |
| TCTCGGCACCACGGGCGTCGTGGGCATCATCAAAGCGGGCACCTCCGACCG | |
| TGCTATTGCCTTTGCGAGCCGATATGGACGCGCTTCCCATGCAGGAA | |
| Code: EAA12: | |
| ATCACACCGGAAGGCCATATTTTTGGGTCGTTACAGCAAGAACCAGCCCTTC | SEQ ID NO 30 |
| AGCCTCGGCGGTGAAAGCACCGTGCATACCGCTGGCAAAGGCGTGACCGTC | |
| GTCGAGTGGCAGGGCATCAAGATTGCACCGCTCATCTGCTATGATCTGCGCT | |
| TTCCGGAGCTCGCTCGCGAGGCCGTGAAGGCCGGCGCCGAGCTGCTCGTCT | |
| TCATCGCCGCGTGGCCGATCAAACGCGTGCAGCATTGGATCACGCTGCTGC | |
| AAGCCCGTGCGATCGAAAACCTCGCGTTCGTCATCGGCGTGAACCAATGCG | |
| GCACCGATCCGAGCTTCACATATCCCGGGCGCAGCCTCGTCGTCGATCCGCA | |
| CGGCGTCATCATCGCCGATGCGGGCGATCACGAGCACGTCCTGCGTGCCGA | |
| GATCGATCCCGCCATCCTCCACGCCTGGCGCAGCCAGTTCCCCGCCTTGCGT | |
| GACGCGGGAATCGCGTCG | |
| Code: EAA13: | |
| ATGAAACTGATCCCCGAAATCCAGGCCGCTCAAGGCGAGATACAAACCCTC | SEQ ID NO 31 |
| CGACGAACGTTCACGCCCACCCAGAACTGCGTTACGAAGAAACTCAGACA | |
| TCCGACCTGGTCGCGAAGAGTTTGAGCGACTGGGGTATCGAGGTGCATCGT | |
| GGGCTCGGCAAAACCGGGGTTGTGGGCATTCTGAAGCGTGGCAGCAGCGAG | |
| CGGGCAATAGGCCTGAGGGCCGACATGAACGCCCTGCCGATCCACGAATTG | |
| AACAGCTTCGAGCATCGTTCACGCCACGAAGGAATGT | |
| Code AA3: | |
| CATTGCCGTATGGCCATCRTGNCCRCA | SEQ ID NO. 32 |
| Code AA4: | |
| GGCCGTGTGGCCTCRTGNCCRCA | SEQ ID NO. 33 |
| Code oli10: | |
| AAGGGTGCCAACCTCTTCAAGGG | SEQ ID NO. 34 |
| Code oli11: | |
| CTTGAAGAGGTTGGCACCCT | SEQ ID NO. 35 |
| Code Am508: | |
| GATATTTAATATGTTTAGCTGCATCAATTCKRAANCCRTC | SEQ ID NO. 36 |
| Code Am510: | |
| GGCGGCGTCGATCCKRAANCCRTC | SEQ ID NO. 37 |
| Code Am14: | |
| GATCAACTTAATTAGCAACATCCATTCKCCANCCRTC | SEQ ID NO. 38 |
| Code Am30: | |
| GCCCCGCTGGGTGTCRTGRTTNTC | SEQ ID NO. 39 |
| Code Am1: | |
| GCATGTTATGCTGGATGCAGTNTTYAAYCA | SEQ TD NO. 40 |
| Code Am3: | |
| AAATGTGCAAGTGTATATGGATTTTGTNYTNAAYCA | SEQ ID NO. 41 |
| Code: EAA1: | |
| NRGMGTTGVVGIVKAGTSERAIALRADMDALPTQEFNTFEHASQHPGK | SEQ ID NO 42 |
| Code: EAA2: | |
| VVLQFTGRRFTHRGLGTTGVVGIVKAGTSERALALRADMDALPMQECNSFAH | SEQ ID NO 43 |
| TSQYPGK | |
| Code: EAA3: | |
| LHELTAFRRDLHVHPELGFEEVYTSGRVAETLRLCGVDEVHTQIGKTGVVAVIK | SEQ ID NO 44 |
| GKRQSSGKMMGLRADMDALPMAEHNEFTWKSAKSGL | |
| Code: EAA4: | |
| LKPAPPQCYSEMALLSRRRSMIQFPFRRCRMLQKAQEIQEPLVAWRREFHTYPE | SEQ ID NO 45 |
| LGFRESRTAARVAEILTGLGYRVRTGVGRTGVVAERGEGHPIIAVRADMDALPI | |
| QEANDVPYASQH | |
| Code: EAA5: | |
| LPELLDQADAMRALRRDIHAHPELCFQEVRTSDLIAKTLQSWGIEVHTGLGTTG | SEQ ID NO 46 |
| VVGVIKGRPGKRAIGLRADIDALPMTEHNTFAHASRHACKTTAQG | |
| Code: EAA6: | |
| GDALTERVGEFLQLRRDIHRHPELAFEEHIRTSELVAAKLESWGYAVRRGLGGT | SEQ ID NO 47 |
| GVVGVLKRGHSQRSLGIRADMDALPIQE | |
| Code: EAA7: | |
| PSLPPSVLPELLDQADAMRALRRDIHAHPELCFQEVRTSDLIAKTLQSWGWVHT | SEQ ID NO 48 |
| GLGTTGVVGVIKGRPGKRAIGLRADDALPMTEHNTFAHSRHAGR | |
| Code: EAA8: | |
| GIPLHRGMGTTGVVGIVKSGTSDRAIGLRADMDALPMAENTFAHASTHPGK | SEQ ID NO 49 |
| Code: EAA9: | |
| ITEFHPELTAFRRDLHVHPELGFEEVYTSGRVAEGLRLCGVDEVHTQIGKTGVV | SEQ ID NO 50 |
| AVIKGKRQTSGKMIGLRADMDALPMAEHNEFTWKSAKT | |
| Code: am27: | |
| MVARCKAVGVDIYVDAVINHMTGVGSGVGSAGSTYSPYNYPGIYQYQDFHHC | SEQ ID NO 51 |
| GRNGNDDIQNYGDRYEVQNCELVNLADLDTGSSYVRDRLAAYLNDLI | |
| Code: am80: | |
| ICLAASIRKPSNHKYDVEDYTSIDPHLGGEAGLLLLREVLDERAMKLVLDIVPN | SEQ ID NO 52 |
| HCGVTHPWFVAAQANPRSPTAEFFMFRRHPDDYESWLGVKTLPKLNYRSVRL | |
| RDVMYAGQDAIMRYWLRP | |
| Code: am156: | |
| RKPEEDNRPLNYRELAHELAEHXKDCGFTHVELLP | SEQ ID NO 53 |
| Code: am159: | |
| TAATSTPTLTITPTTSPIDKPEWWKSAVFYQWVFVRXFYDSDGDGIGDFQGLIQKL | SEQ ID NO 54 |
| DYLNDGDPKTNSDLGINAVWLMPVNPSPSYHGYDVTDYYNVNPDYGTMDDF | |
| RELIKEAHORGIKVIIDLVINHTSTQHPWFQQALDPQSPYHNYYIWRDENPGYS | |
| GPDGQKVWHRASNGKYYYALFWDQMPDLNFQNPQVTEEIYQIARFWLEDVG | |
| VD | |
| Code: am161: | |
| YNDNISTAGPFNELPSPALKVTLVGLGYRLNNQTFYPDYQSEVMGAVSLVRRM | SEQ ID NO 55 |
| FPLANSAGGSGLAWDYWHIMDEGLGSRVNMTNVECNDYISWEDGKVVDRRN | |
| LCSTRYANHLLAYLRSAWKYSDRLFAYGLISTN | |
| Code: am162: | |
| MIGYEIFVRSFADSNDDGIGDFKGIAQKVDYFKMLGVDLIWLTPHFKSPSYHGY | SEQ ID NO 56 |
| DIIDYFDTNVSFGTLADFRDMVDKLHANGIKIVIDLPFNHVSDRHPWFKAAMN | |
| GEKPYVDYFLWAQPHFNLKEKRHWDEELLWHTRNGKTYYGVFGGSSPDLNY | |
| ENPEVVQN | |
| Code: am163: | |
| RETPILQWFQTDYRTILQRLPEVVQAGYGAIYLPSPVKSGGGGFSTGYNPFDLFD | SEQ ID NO 57 |
| LGDRFQKGTVRTQYGTTQELIELIRLAQRLGLEVYCDLVTNHAD | |
| Code: am164: | |
| MSDTEKPRRTRRKQVANTDEPSTTVTASTTDAPTATIEEPSAAARAMMTSILSE | SEQ ID NO 58 |
| DDIYLFNQGTHYRLYDKFGAQPVVLEGVPGTYFAVWAPNAEYVAVIGDWNN | |
| WDAGANPLRQRGFSGVWEGFIPHVGKGMRYKIFHIASRYYGYREDKTDPFGTY | |
| FEVAPQTAAIIWDRDYTWS | |
| Code: am170: | |
| SSLPFGPVHHSTARAQTSSPRTVFVHLFEWKWTDIAQECENFLGPRGFAAVQVS | SEQ ID NO 59 |
| PPQEHAIVAGYPWWQRYQPVSYQLTSRSGTRAEFANMVARCKAVGVDIYVDA | |
| VINHMTGVGSGVGSAGSTYSPYNYPGIYQYQDFHHCGRNGNDDIQNYGDRYE | |
| VQNCELVNLADLDTGSSYVRDRLAAYLNDLIM | |
| Code: am173: | |
| LFPEKLGAHPTEIDGVKGVYFAVWAPNARNVSVIGDFNQWDGRKHQMRKGQT | SEQ ID NO 60 |
| GVWELFTPELGVGEHYKYEJKNLEGHIYEKSDPYGFQQEPRPKTASIVTDLNSYQ | |
| WNDEDWMEQRRHTYPLTQPISVYEVHLGSWLHASSAEPPRLPNGETEPVVPVS | |
| ELNPGARFLTYRELADRLIPYVKDLGYTHVELLPIAEHPFDGSWGYQVTGYYAP | |
| TSRYGSPEDFMYFV | |
| Code: am159-G: | |
| MKLTRLRHITVLIIILSLLGACTTPQKPSNEGAAATSTPTLTITPTTSPIDKPEWWK | SEQ ID NO 61 |
| SAVFYQVFVRSFYDSDGDGIGDFQGLIQKLDYLNDGDPKTNSDLGINAVWLMP | |
| VNPSPSYHGYDVTDYYNVNPDYGTMDDFRELIKEAHQRGIKVIIDLVINIHTSTQ | |
| HPWEQQALDPQSPYHNYYTWRDENPGYSGPDGQKVWHRASNGKYYYALFWD | |
| QMPDLNFQNPQVTEEIYQIARFWLEDVGVDGFRIDAAKHLIEEGTDQENTGLTH | |
| EWFASFYQYYKSLNPQAVTVGEVWSNSFEAVRYVRNQEMDMVFNFDLARSIX | |
| TXINNRNAVSLSNTLTFEXRLFPKGSMGIFXTNHDQDRVMTVLMNDEQKARLX | |
| AAVYXTSPGVPFIYYGEEIGLTGQGDHRNLRTPMHWSAERMAGFTSGTPWLFP | |
| KMDYAEKNVEDQLEDPNSLLRFYMDLLRIRSQSKALQSGELSALSSSSSSIILAY | |
| ARVSQNEQVLIVLNLGNQPQERVTLHSVEGLNPGTYRLSPLLGGQVNTTIIVEP | |
| DGALQEFEFPATISANEVLIYQLINSTE | |
| Code: am162-G: | |
| MIGYEIIFVRSFADSNIDDGIGDFKGJAQKVDYFKMLGVDLIWLTPHFKSPSYUGY | SEQ ID NO 62 |
| DIIDYEDTNVSFGTLADFRDMVDKLHANGIKIVIDLPFNHVSDRHPWFKAAMN | |
| GEKPYVDYFLWAQPIWNLKEKRHWDEELLWHTRNGKTYYGVFGGSSPDLNY | |
| ENPEVVQKSLEIVEFWLKQGVDGPRFDAAKHILYDYDIKEGKFRYDHEKNVAY | |
| WQLVMDRARQIKGEDVFAVTEVWDDPEIVDRYAKTIGCSFNFYFTEAIRESMQ | |
| HGAVYKIVDCFQRTLTKKPYLPSNIFTGNHDMHRLAQLLPHEEQRKVFFGLLMT | |
| TPGVPFIYYGDELGMKGQYDSTFTEDVTEPFPWYASLSGEGQAFWKAVRFNRA | |
| FTGASVEEHLNREDSLLKEVINWTKFRKENDWLTNAWVEHVTHNTFTIAYTFVT | |
| DGDNGFRVYVNIAGIHIHIETFEGVSLKAYEVKVL | |
| Code: am164-G: | |
| MSDTEKPRRTRRKQVANTDEPSTTVTASTTDAPTATIEEPSAAARAMMTSILSE | SEQ ID NO 63 |
| DDIYLFNQGTHYRLYDKFGAQPVVLEGVPGTYFAVWAPNAEYVAVIGDWNN | |
| WDAGANPLRQRGFSGVWEGFIPHVGKGMRYKFHLASRYYGYREDKTDPFGTY | |
| FEVAPQTAAIIWDRDYTWSDQQWMSERGQRQRLDAPISIYEVHLGSWRRKPEE | |
| DNRPLNYRELAHELVEHVKDCGFTHVELLPVTEHPFYGSWGYQSTGLFAPTSR | |
| YGTPQDFMYFVDYLHQNGIGVILDWVPSTWPTDGHGLAYFDGTHLYEHADPR | |
| KGYHPDWGSYIYNYGRNEVRSFLISSALCWLDKFHIDGIRVDAVASMLYLDYS | |
| RRAGEWIPNEYGGNENLEAISFLRELNTQIYKYYPDVQTIAEESTAWPMVSRPV | |
| YVGGLGFGFKWDMGWMHIDTLQYFRRDPIYRRFHHNELTFRGLYMIFSENYVLP | |
| LSHDEVVHGKGSLLDKMAGDVWQKFANLRLLYSYMFAQPGKKLLFMGGEFG | |
| QWREWSHDTSLDWIILLMFPSHQGVQRLIGDLNRLYRTEPALHELDCDPRGFE | |
| WIDANDADASVYSFLRKSRYGEQILIVINATPVVREDYRIGVPVGGWWRELFNS | |
| DSEYYWGSGQGNAGGVMAEAIPTHGRDFSLRLRLPPLGALFLKPAG | |
| Code: am170-G: | |
| MPGTRFPSLRRLVLVVALLMVVSSLPFGPVHHSTARAQTSSPRTVFVHLFEWK | SEQ ID NO 64 |
| WTDIAQECENPLGPRGFAAVQVSPPQEHAIVAGYPWWQRYQPVSYQLTSRSGT | |
| RAEXPHMVARCKAVGVDIYVDAVINHMTGVGSGVGSAGSTYSPYNYPGIYQY | |
| QDFFHHCGRNGNDDIQNYGDRYEVQNCELVNLADLDTGSSYVRDRLAAYLNDL | |
| ISLGVAGFRIDAAKHIAAGDIAAILSRVNGSPYIYQEVIGAAGEPITPWEYTNNG | |
| DVTEFKYSNEIGRVFLNGKLAWLSQFGEAWGMILPSDKAIVFVDNHIDNQRGHG | |
| GGGTVVTYKNGVLYDLANVFMLAWPYGYPQVMSSYEFSNDFQGPPSDANGN | |
| TRSVYVNXQPNCFGEWKCEHRWRPLANMVAFRNATASTFSVSDWWSNGNNQI | |
| AFGRGDKGFVVINREDTTLNRTFOTSMAPGVYCNVIVADFTNGTCSGQTVTVD | |
| SNRRITVSIPPFSALAIHVGAKLSTQPATVAVTFNVNATTYWGQNVFVVGNIPQ | |
| LGNWNPAQAVPLSAATYPVWSGTVNLPANTTIEYKYIKRDGSNVVWECCNNR | |
| VITTPGSGSMTLNETWRP | |
| Code: am80: | |
| TDLGVSALYLNPIFRAPSNHKYDVEDYTSIDPHLGGEAGLLLLREVLDERAMKL | SEQ ID NO 65 |
| VLDIVPNHCGVTHPWFVAAQANPRSPTAEFFMFRRHPDGYESWLGVKTLPKLN | |
| YRSVRLRDVMYAGQDAIMRYWLRPPYRI | |
| Code: am81: | |
| ADCLISDYSDRYQVQYCQLAGLPDLDTGKSTVQTKLRAYLQALLNAGVKGFRI | SEQ ID NO 66 |
| DAAKUMAAHEVGAILDGLTLPGGGRPYIFSEVIDMDPNERIRDWEYTPYGDVT | |
| EFAYSISVIGNTFNCGGSLSNLQNFJTNLLPSHFAQIFVDNIHDTQRGKGEFV | |
| Code: am82: | |
| GEIVDPSDVQMAFAGQLDGALDFILLEGLRQAIAFGRWNGFQLASFLERHQIYF | SEQ ID NO 67 |
| PEDFSRPSFLDNHDTQRGKG | |
| Code: am103: | |
| DFHADCLISDYSDRYQVQYCQLAGLPDLDTGKSTVQTKLRAYLQALLNAGVK | SEQ ID NO 68 |
| GFRIDAAKHMAAHEVGAILDGLTLPGGGRPYIFSEVIDMDPNERIRDWEYTPYG | |
| DVTEFAYSLSVIGNTFNCGGSLSNLQNFITNLLPSHEAQIPVDNHDTQRGKG | |
| Code: EAA10: | |
| MKLTDSIVQNTPTIAAVRRDLHAHPELCFEENRTADKVASKLAEWGIPFHRGLA | SEQ ID NO 69 |
| TTGVVGIIQSGTSDRAIGLRADMDALPMQEVNT | |
| Code: EAA11: | |
| MNLIDSIVSSAASIAAVRRDLFIAHPELCFKEVHTSDVVAQRLTDWGIPIIHRGLG | SEQ ID NO 70 |
| TTGVVGIIKAGTSDRAIALRADMDALPMQE | |
| Code: EAA12: | |
| ITPEGLHLGRYSKNQPFSLGGESTVHTAGKGVTVVEWQGIKIAPLICYDLRPPEL | SEQ ID NO 71 |
| AREAVKAGAELLVFIAAWPIKRVQHWITLLQARAIENLAFVIGVNQCGTDPSFT | |
| YPGRSLVVDPHGVIIADAGDHEHVLRAEIDPAWHAWRSQFPALRDAGIAS | |
| Code: EAA13: | |
| MKLJPEIQAAQGEIQTLRRTIHAHPELRYEETQTSDLVAKSLSDWGTEVHRGLGK | SEQ ID NO 72 |
| TGVVGILKRGSSERAIGLRADMNALPTHIELNSFEHRSRHEGM | |

### REFERENCES

Aevarsson, A., Marteinsson, V. T., Hreggvidsson, G. O., Kristjansson, J. K. and Fridjonsson, O. H.: Method of obtaining protein diversity, U.S. patent application Ser. No. 09/878,423. Prokaria ltd, 2001.

Altschul, S. F., Gish, W., Miller, W., Myers, E. W. and Lipman, D. J.: Basic local alignment search tool. J Mol Biol 215 (1990) 403-410.

Altschul, S. F., Madden, T. L., Schaffer, A. A., Zhang, J., Zhang, Z., Miller, W. and Lipman, D. J.: Gapped BLAST and PSI-BLAST: a new generation of protein database search programs. Nucleic Acids Res 25 (1997) 3389-3402.

Anders, M. W. and Dekant, W.: Aminoacylases. Adv Pharmacol 27 (1994) 431-448.

Antranikian, G.: Physiology and enzymology of thermophilic anaerobic bacteria degrading starch. FEMS Microbiol Lett 75 (1990) 201-218.

Ausubel, F. M. et al., “Current Protocols in Molecular Biology”, John Wiley & Sons, (1998).

Bateman, A., Birney, E., Durbin, R., Eddy, S. R., Finn, R. D. and Sonnhammer, E. L.: Pfam 3.1: 1313 multiple alignments and profile HMMs match the majority of proteins. Nucleic Acids Res 27 (1999) 260-262.

Dalboge, H.: Expression cloning of fungal enzyme genes; a novel approach for efficient isolation of enzyme genes of industrial relevance. FEMS Microbiol Rev 21 (1997) 29-42.

Henikoff, S., Henikoff, J. G., Alford, W. J. and Pietrokovski, S.: Automated construction and graphical presentation of protein blocks from unaligned sequences. Gene 163 (1995) 17-26.

Henne, A., Schmitz, R. A., Bomeke, M., Gottschalk, G. and Daniel, R.: Screening of environmental DNA libraries for the presence of genes conferring lipolytic activity on Escherichia coli. Appl Environ Microbiol 66 (2000) 3113-3116.

Henrissat, B. and Davies, G.: Structural and sequence-based classification of glycoside hydrolases. Curr Opin Struct Biol 7 (1997) 637-644.

Jones, D. H. and Winistorfer, S. C.: Sequence specific generation of a DNA panhandle permits PCR amplification of unknown flanking DNA. Nucleic Acids Res 20 (1992) 595-600.

Jones, D. H. and Winistorfer, S. C.: A method for the amplification of unknown flanking DNA: targeted inverted repeat amplification. Biotechniques 15 (1993) 894-904.

Karlin et al., Proc. Natl. Acad. Sci. U.S.A., 90 (1993) 5873-5877.

Kilstrup, M. and Kristiansen, K. N.: Rapid genome walking: a simplified oligo-cassette mediated polymerase chain reaction using a single genome-specific primer. Nucleic Acids Res 28 (2000) E55.

Krause, M. H. and S. A. Aaronson, Methods in Enzymology, 200:546-556 (1991).

Laging, M., Fartmann, B. and Kramer, W.: Isolation of segments of homologous genes with only one conserved amino acid region via PCR. Nucleic Acids Res 29 (2001) E8.

Maidak, B. L., Cole, J. R., Parker Jr, C. T., Garrity, G. M., Larsen, N., Li, B., Lilburn, T. G., McCaughey, M. J., Olsen, G. J., Overbeek, R., Pramanik, S., Schmidt, T. M., Tiedje, J. M. and Woese, C. R.: A new version of the RDP (Ribosomal Database Project). Nucleic Acids Res 27 (1999) 171-173.

Marteinsson, V. T., Hobel, C., Fridjonsson, O. H., Hreggvidsson, G. O. and Kristjansson, J. K.: Accessing microbial diversity by ecological methods, U.S. patent application Ser. No. 09/770,771. Prokaria ltd, 2001a.

Marteinsson, V. T., Kristjansson, J. K., Kristmannsdottir, H., Dahlkvist, M., Saemundsson, K., Hannington, M., Petursdottir, S. K., Geptner, A. and Stoffers, P.: Discovery and description of giant submarine smectite cones on the seafloor in Eyjafjordur, northern Iceland, and a novel thermal microbial habitat. Appl Environ Microbiol 67 (2001b) 827-833.

Megonigal, M. D., Rappaport, E. F., Wilson, R. B., Jones, D. H., Whitlock, J. A., Ortega, J. A., Slater, D. J., Nowell, P. C. and Felix, C. A.: Panhandle PCR for cDNA: a rapid method for isolation of MLL fusion transcripts involving unknown partner genes. Proc Natl Acad Sci USA 97 (2000) 9597-9602.

Morris, D. D., Gibbs, M. D., Chin, C. W., Koh, M. H., Wong, K. K., Allison, R. W., Nelson, P. J. and Bergquist, P. L.: Cloning of the xynB gene from Dictyoglomus thermophilum Rt46B.1 and action of the gene product on kraft pulp. Appl Environ Microbiol 64 (1998) 1759-65.

Radomski, C. C. A., Seow, K. T., Warren, R. A. J. and Yap, W. H.: Method for isolating xylanase gene sequences from soil DNA, compositions useful in such method and compositions obtained thereby, U.S. Pat. No. 5,849,491. Terragen Diversity Inc., 1998.

Rawlings, N. D. and Barrett, A. J.: Evolutionary families of metallopeptidases. Methods Enzymol 248 (1995) 183-228.

Riley, J., Butler, R., Ogilvie, D., Finniear, R., Jenner, D., Powell, S., Anand, R., Smith, J. C. and Markham, A. F.: A novel, rapid method for the isolation of terminal sequences from yeast artificial chromosome (YAC) clones. Nucleic Acids Res 18 (1990) 2887-2890.

Rondon, M. R., Raffel, S. J., Goodman, R. M. and Handelsman, J.: Toward functional genomics in bacteria: analysis of gene expression in Escherichia coli from a bacterial artificial chromosome library of Bacillus cereus. Proc Natl Acad Sci U S A 96 (1999) 6451-6455.

Rose, T. M., Schultz, E. R., Henikoff, J. G., Pietrokovski, S., McCallum, C. M. and Henikoff, S.: Consensus-degenerate hybrid oligonucleotide primers for amplification of distantly related sequences. Nucleic Acids Res 26 (1998) 1628-1635.

Rosenthal, A. and Jones, D. S.: Genomic walking and sequencing by oligo-cassette mediated polymerase chain reaction. Nucleic Acids Res 18 (1990) 3095-3096.

Rubie, C., Schulze-Bahr, E., Wedekind, H., Borggrefe, M., Haverkamp, W. and Breithardt, G.: Multistep-touchdown vectorette-PCR—a rapid technique for the identification of IVS in genes. Biotechniques 27 (1999) 414-6, 418.

Short, J. M.: Protein activity screening of clones having DNA from uncultivated microorganisms, U.S. Pat. No. 5,958,672. Diversa Corporation, 1999.

Shyamala, V. and Ames, G. F.: Genome walking by single-specific primer polymerase chain reaction: SSP PCR. Gene 84 (1989) 1-8.

Skirnisdottir, S., Hreggvidsson, G. O., Hjorleifsdottir, S., Marteinsson, V. T., Petursdottir, S. K., Holst, O. and Kristjansson, J. K.: Influence of sulfide and temperature on species composition and community structure of hot spring microbial mats. Appl Environ Microbiol 66 (2000) 2835-2841.

Sorensen, A. B., Duch, M., Jorgensen, P. and Pedersen, F. S.: Amplification and sequence analysis of DNA flanking integrated proviruses by a simple two-step polymerase chain reaction method. J Virol 67 (1993) 7118-7124.

Stokes, H. W., Holmes, A. J., Nield, B. S., Holley, M. P., Nevalainen, K. M., Mabbutt, B. C. and Gillings, M. R.: Gene cassette PCR: sequence-independent recovery of entire genes from environmental DNA. Appl Environ Microbiol 67 (2001) 5240-5246.

Takehiko, Y.: Enzyme chemistry and molecular biology of amylases. In: Takehiko, Y., Sumio, K., Seiya, C., Keitaro, H., Yoshiki, M., Noshi, M., Yasunori, N., Ryu, S. and Kunio, Y. (Eds.), Enzyme chemistry and molecular biology of amylases and related enzymes. CRC Press, Boca Raton, Fla., 1995, pp. 81-100.

Thompson, J. D., Higgins, D. G. and Gibson, T. J.: CLUSTAL W: improving the sensitivity of progressive multiple sequence alignment through sequence weighting, position-specific gap penalties and weight matrix choice. Nucleic Acids Res 22 (1994) 4673-4680.

Woo, S. S., Jiang, J., Gill, B. S., Paterson, A. H. and Wing, R. A.: Construction and characterization of a bacterial artificial chromosome library of Sorghum bicolor. Nucleic Acids Res 22 (1994) 4922-4931.

Zhou, M. Y. and Gomez-Sanchez, C. E.: Universal TA cloning. Curr Issues Mol Biol 2 (2000) 1-7.

We claim:

1. A method for obtaining at least one specific DNA sequence related to a target sequence, from a sample comprising a mixed population of a plurality of microbial species, comprising DNA or a mixture of nucleic acids, the method comprising:
a) extracting the DNA or mixture of nucleic acids from said sample;
b) hybridizing said DNA or mixture of nucleic acids with a degenerate primer targeted to a single region in said target sequence to synthesize at least one single stranded copy-DNA complementary to a region of said target sequence, said synthesis being primed by said degenerate primer and catalyzed by a DNA-polymerase or a reverse transcriptase; and performing a linear amplification of said at least one single stranded copy-DNA by repeated thermal cycling;
c) purifying the single stranded copy-DNA synthesized in step b);
d) providing a second primer site to the 3′ end of the single stranded copy-DNA; and
e) amplifying the single stranded copy-DNA using a primer pair wherein a first primer comprises at least a part of the degenerate primer sequence and a second primer which is complementary to the 3′ primer site of step d) or is an arbitrary primer;
to thereby obtain at least one specific DNA sequence related to said target sequence.

2. The method according to claim 1 wherein said second primer site is provided by a method selected from the group consisting of:
a) ligating an anchor sequence to the 3′ end of the purified single stranded copy-DNA;
b) producing an anchor sequence by successively adding nucleotides to the 3′ end of the purified single stranded copy-DNA by use of terminal DNA transferase;
c) using an arbitrary primer;
d) ligating a double stranded oligonucleotide adaptor to a fragmented target DNA, following enzymatic restriction or mechanical treatment prior to generation of single stranded DNA; and
e) ligating fragmented targeted DNA following enzymatic restriction or mechanical treatment to vector DNA.

3. The method according to claim 2, wherein said ligation of the 3′ anchor sequence of step (a) is catalyzed by a single strand-DNA ligating enzyme such as T4 RNA ligase.

4. The method according to claim 1, wherein the degenerate primer of step (b) is additionally used as an arbitrary reverse primer in the amplification reaction of step e).

5. The method according to claim 1, wherein the amplification of in step (e) is performed by an amplification method that is dependent on a 5′ located and a 3′ located primer.

6. The method according to claim 5, wherein the amplification step is performed by a n amplification method selected from the group consisting of polymerase chain reaction (PCR), nucleic acid sequence based amplification (NASBA) and strand displacement amplification (SDA).

7. The method according to claim 5, wherein the amplification step is performed by PCR.

8. The method according to claim 1, wherein said degenerated primer comprises a short 3′ degenerate core region in the range from about 8 to about 15 nucleotides, and a longer 5′ consensus clamp region in the range from about 12 to about 30 nucleotides.

9. The method according to claim 1, wherein said degenerated primer at its 5′ end is labeled with one member of an affinity pair.

10. The method according to claim 9, wherein the affinity pair is selected from the group consisting of biotin—streptavidin, biotin—avidin, digoxigenin—anti-hapten antibody, fluorescein—anti-hapten antibody, lectins—lectin receptor, ion-ion chelators, IgG—protein A, IgG—protein G and magnets—paramagnetic particles.

11. The method of claim 1, further comprising amplifying flanking regions to said DNA sequence to obtain a functional gene comprising said DNA sequence.

12. The method of claim 11, wherein said flanking regions are amplified with one or more steps of nested PCR reactions.

13. The method of claim 1, further comprising screening said sample or a DNA library derived from said sample to isolate a functional gene encoding a protein, using a probe having a sequence which is the same as or complementary to at least a portion of said obtained DNA sequence.

14. The method according to claim 1, wherein said sample of DNA or nucleic acids is a complex mixture of nucleic acids extracted from mixed cultures of microorganisms.

15. The method according to claim 1, wherein said sample of DNA or nucleic acids is a complex mixture of nucleic acids extracted from an environmental sample.

16. The method according to claim 15, wherein the environmental sample is derived from an oligotrophic environment.

17. The method according to claim 15, wherein the environmental sample is derived from an extreme environment.

18. The method according to claim 15, wherein the environmental sample is derived from a terrestrial geothermal environment.

19. The method according to claim 15, wherein the environmental sample is derived from a marine geothermal environment.

20. The method according to claim 1 wherein the sample is enriched for a microbial population by maintaining the sample under conditions substantially similar to the environment from which the sample was obtained to thereby expand the microbial population; and allowing a sufficient quantity of a microbial population to expand; whereby the population has been enriched.

21. A method for obtaining a functional gene encoding an aminoacylase/amidohydrolase from a sample comprising DNA and/or a mixture of nucleic acids, comprising screening said sample using a nucleic acid probe comprising a nucleotide sequence which is selected from the group consisting of:
a) SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:28, SEQ ID NO:29, SEQ ID NO:30, and SEQ ID NO:31;
b) a nucleotide sequence encoding a polypeptide comprising a sequence selected from the group consisting of SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:69, SEQ ID NO:70, SEQ ID NO:71, and SEQ ID NO:72;
c) a nucleotide sequence that encode a polypeptide having at least 75% sequence identity to a polypeptide of step b); and
d) a nucleotide sequence that is complementary to a nucleotide sequences of step a), b), or c).

22. A method for obtaining a functional gene encoding an amylase from a sample comprising DNA and/or a mixture of nucleic acids, comprising screening said sample using a nucleic acid probe comprising a nucleotide sequence selected from the group consisting of:
a) SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:20, SEQ ID NO:21, SEQ ID NO:22, SEQ ID NO:23, SEQ ID NO:24, SEQ ID NO:25, SEQ ID NO:26, and SEQ ID NO:27;
b) a nucleotide sequence encoding a polypeptide comprising a sequence from the group of SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:61, SEQ ID NO:62, SEQ ID NO:63, SEQ ID NO:64, SEQ ID NO:65, SEQ ID NO:66, SEQ ID NO:67, and SEQ ID NO:68;
c) a nucleotide sequence that encodes a polypeptide having at least 65% sequence identity to a polypeptide sequence listed in b); and
d) a nucleotide sequence that is complementary to a sequences of step a), b), c).

23. A method for obtaining a functional gene encoding an amylase from a sample comprising DNA and/or a mixture of nucleic acids, comprising screening said sample using a nucleic acid probe comprising a nucleotide sequence from the group consisting of SEQ ID NO: 19; sequences encoding the polypeptide described by SEQ ID NO:60; sequences encoding polypeptides having at least 80% sequence identity to SEQ ID NO:60; and sequences that are complementary to any of said sequences.

24. An isolated nucleic acid molecule having a nucleic acid sequence which is part of a gene encoding for an aminoacylase/amidohydrolase, selected from the group consisting of:
a) SEQ ID NO:1 and SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:29; and SEQ ID NO:30;
b) sequences encoding a polypeptide comprising a sequence from the group consisting of SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:70, and SEQ ID NO:71;
c) and sequences encoding polypeptides having at least 65% sequence identity with a polypeptide encoded by any of said sequences; and
d) sequences that are complementary to any of said nucleotide sequences of a)-c).

25. An isolated nucleic acid molecule having a nucleic acid sequence which is part of a gene encoding an aminoacylase/amidohydrolase, selected from the group consisting of SEQ ID NO:28 and SEQ ID NO:31; and sequences encoding polypeptides having at least 75% sequence identity with a sequence from SEQ ID NO:69 and SEQ ID NO:72.

26. An isolated nucleic acid molecule encoding an aminocylase/amidohyrolase, comprising a nucleic acid sequence of claim 24.

27. An isolated nucleic acid molecule encoding an aminocylase/amidohyrolase, comprising a nucleic acid sequence of claim 25.

28. An isolated polypeptide encoded by the sequence of claim 26.

29. An isolated polypeptide encoded by the sequence of claim 27.

30. An isolated nucleic acid molecule having a nucleic acid sequence which is part of a gene encoding for an amylase, said sequence selected from the group consisting of:
a) SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:20, SEQ ID NO:21, SEQ ID NO:22, SEQ ID NO:23, SEQ ID NO:24, SEQ ID NO:25, SEQ ID NO:26, and SEQ ID NO:27;
b) sequences encoding a polypeptide comprising a sequence from the group of SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:61, SEQ ID NO:62, SEQ ID NO:63, SEQ ID NO:64, SEQ ID NO:65, SEQ ID NO:66, SEQ ID NO:67, and SEQ ID NO:68;
c) sequences encoding for polypeptides having at least 65% sequence identity to a polypeptide sequence listed in b); and
d) sequences that are complementary to any of said sequences of a)-c).

31. An isolated nucleic acid sequence which sequence is part of a gene encoding for an amylase, said sequence from the group consisting of SEQ ID NO:19; and sequences encoding for the polypeptide described by SEQ ID NO: 60; and sequences encoding for polypeptides having at least 80% sequence identity to SEQ ID NO:60.

32. An isolated nucleic acid molecule encoding for an amylase, comprising a nucleic acid sequence of claim 30.

33. An isolated nucleic acid molecule encoding for an amylase, comprising a nucleic acid sequence of claim 31.

34. An isolated polypeptide encoded by the nucleic acid molecule of claim 32.

35. An isolated polypeptide encoded by the nucleic acid molecule of claim 33.


SEQUENCE LISTING (72 sequences)

SEQ ID NO: 1 - DNA, 144 bp, Unknown
  DNA retrieved from environmental DNA; Aminoacylase/Amidohydrolase genes;EAA1
aaccggggcatgggtaccaccggcgttgtcggaatcgtgaaagccggcacgtcggagcgcgccattgccctgcgtgccgacatggacgccttgccgacgcaggagttcaacacttttgagcacgccagccaacaccctggaaag

SEQ ID NO: 2 - DNA, 180 bp, Unknown
  DNA retrieved from environmental DNA; Aminoacylase/Amidohydrolase genes; EAA2
tgagtcgtattacaattcactggccgtcgttttacacaccgtggtttgggtactaccggcgtcgtcggcatcgtgaaggcaggcacctcggaacgtgcactggccttgcgcgcggatatggatgccctgcccatgcaagagtgcaacagctttgcccacaccagccaatacccaggcaag

SEQ ID NO: 3 - DNA, 270 bp, Unknown
  DNA retrieved from environmental DNA; Aminoacylase/Amidohydrolase genes; EAA3
ttacacgaactcacggctttccgccgtgacctgcatgttcaccccgagctggggtttgaagaggtttacactagcgggcgggtcgcagagaccctgcgcctgtgcggtgtggatgaggttcatacgcagattggcaagaccggcgtggtggcggttatcaaaggcaagcgtcaaagcagcggcaagatgatggggctgcgtgccgacatggacgcgctaccgatggccgagcacaacgagttcacctggaaatctgccaaatccggcctg

SEQ ID NO: 4 - DNA, 362 bp, Unknown
  DNA retrieved from environmental DNA; Aminoacylase/Amidohydrolase genes; EAA4
ctaaagcccgcccctccccaatgctacagcgaaatggctctgttgtcaaggaggcgcagtatgatacaattccccttcaggaggtgccggatgctccaaaaagcgcaggagattcaagaacccctggtggcctggcgacgggagtttcacacttaccctgaactgggcttccgggagagccgtacagccgcccgggtggccgaaattttgaccggactgggctatcgcgtccggacgggcgttgggcggaccggagtggtggcggagcggggggaggggcaccccattattgccgtgcgcgccgatatggatgccctgccgatccaggaggccaacgacgtcccctatgcctctcagcaccc

SEQ ID NO: 5 - DNA, 298 bp, Unknown
  DNA retrieved from environmental DNA; Aminoacylase/Amidohydrolase genes; EAA5
ctgcctgaactgctggaccaggccgatgccatgcgggctttgcggcgcgacatccatgcgcaccccgagctgtgttttcaagaagtacgcacctcagacctgatcgccaagaccttgcaaagctggggcattgaggtgcacacgggtctgggcacgaccggtgtcgtgggcgtgatcaaagggcgccccggcaagcgggccattggcttgagggcagacatcgacgccctgcccatgaccgagcacaacacctttgcccatgccagccgacacgcgtgtaaaacgacggcccagggaa

SEQ ID NO: 6 - DNA, 244 bp, Unknown
  DNA retrieved from environmental DNA; Aminoacylase/Amidohydrolase genes; EAA6
ggtgacgcgctcaccgaacgagtgggtgagttcatacagctcaggcgtgacattcatcgccaccccgagctggcgtttgaagagcatagaacgtccgagctggtcgctgccaagctggagagctggggctacgcggtgcgtcgcggcctgggtggaaccggagtggtgggtgttttaaagcgcggccacagtcaacgcagtctgggcattcgtgccgacatggacgcgctgcccattcaggagg

SEQ ID NO: 7 - DNA, 305 bp, Unknown
  DNA retrieved from environmental DNA; Aminoacylase/Amidohydrolase genes; EAA7
ccttcgttgccaccttccgtcctgcctgaactgctggaccaggccgatgccatgcgggctttgcggcgcgacatccatgcgcaccccgagctgtgttttcaagaagtacgcacctcagacctgatcgccaagaccttgcaaagctggggcattgaggtgcacacgggtctgggcacgaccggtgtcgtgggcgtgatcaaagggcgccccggcaagcgggccattggcttgagggcagacatcgacgccctgcccatgaccgagcacaacacctttgcccatgccagccgacacgcgggccgcat

SEQ ID NO: 8 - DNA, 157 bp, Unknown
  DNA retrieved from environmental DNA; Aminoacylase/Amidohydrolase genes; EAA8
ggcattcccctccaccgtggcatgggcaccaccggtgtcgtcggtatcgtcaaaagcgggacatctgatcgggctattggattgcgcgctgacatggatgcgctgcctatggctgaagccaacacctttgcgcacgccagcacccacccaggcaaga

SEQ ID NO: 9 - DNA, 276 bp, Unknown
  DNA retrieved from environmental DNA; Aminoacylase/Amidohydrolase genes; EAA9
attaccgagtttcatcccgaactcacggctttccggcgtgacctgcatgttcaccccgagttggggtttgaagaggtctacaccagcgggcgggttgctgagggcttgcgcctgtgcggcgtggatgaggtccatacgcaaattggcaagaccggcgtggtggctgttatcaaaggcaagcgtcaaaccagcggcaagatgatagggctgcgtgccgacatggacgcgctaccaatggccgagcacaacgagttcacctggaaatctgccaagacc

SEQ ID NO: 10 - DNA, 298 bp, Unknown
  DNA retrieved from environmental DNA; Amylase gene; am27
atggttgcccgttgcaaagcggtcggtgttgacatttatgttgatgcggtcatcaatcatatgaccggcgtcggcagcggtgtcggatcggctggctcaacgtatagcccgtacaactatccgggcatctatcaatatcaggattttcaccactgcggcagaaatggcaacgatgacatccagaattatggtgatcggtacgaagttcagaactgcgaactggtgaatcttgccgatctcgataccggatcatcgtatgtgcgggatcgcttagctgcctatttgaacgatctcatca

SEQ ID NO: 11 - DNA, 373 bp, Unknown
  DNA retrieved from environmental DNA; Amylase gene; am80
atatgtttagctgcatcaattcggaaaccgtcaaaccacaaatacgatgtcgaagactataccagcattgaccctcacctgggaggtgaagcagggttactcctcttacgcgaggtactcgacgagcgagccatgaagctggtgcttgacatcgtcccgaaccattgtggagtgacccatccgtggtttgtcgctgcccaggccaacccacgatcaccaacagccgagttcttcatgttccgtcgtcatcccgacgactacgagagctggctgggggtcaagaccctgcccaaactcaattaccgcagtgtccgcctccgcgacgtaatgtacgcaggccaggatgcgattatgcgctactggttgcgaccac

SEQ ID NO: 12 - DNA, 105 bp, Unknown
  DNA retrieved from environmental DNA; Amylase gene; am156
cgcaaaccggaagaggataaccgtccgctcaattaccgtgaactggcccacgagctggccgagcatgngaaagattgtggctttacccacgttgagctgttaccg

SEQ ID NO: 13 - DNA, 640 bp, Unknown
  DNA retrieved from environmental DNA; Amylase gene; am159
acggctgctacatccactcccaccctcacaatcactccgaccactagtccaatagataaaccggaatggtggaaatcggcggttttctatcaggtgtttgtgcgcantttttatgactctgatggagatggaattggcgattttcagggattgattcagaagctggactatttgaatgatggtgatcccaaaacgaacagtgatttggggattaatgccgtttggttgatgcctgttaatccctcgccgtcttatcacgggtacgatgtgaccgattactacaatgtgaatcccgattacggaacgatggatgatttcagggaattgataaaggaggctcatcagcgcggcattaaagtaattattgatttggtgatcaatcatacatctactcagcacccctggtttcaacaggcattagacccccaatctccttaccataattattacatctggcgggacgaaaatccgggttacagcggaccggatggacaaaaggtctggcatcgcgcctcgaatgggaaatattactacgcgcttttctgggatcaaatgcctgacctgaacttccagaatccgcaggtcactgaggaaatttatcagatcgctcgtttctggctggaagatgtgggtgtggacg

SEQ ID NO: 14 - DNA, 411 bp, Unknown
  DNA retrieved from environmental DNA; Amylase gene; am161
tacaacgacaacatatccaccgccggaccgttcaacttcctgccttcgcccgcgctcaaagtgacgctggttggtctggggtatcggctcaacaatcagactttctatcccgactatcagagtgaggtgatgggtgccgtctcactggtgcggcgaatgttccccctggccaactcagccggtggatcaggtctcgcctgggattactggcacatcatggatgaaggactcggctcgcgtgtgaacatgaccaatgtcgagtgtaacgattatatctcgtgggaagacggcaaggtggtggatcggcgtaacctgtgttcgacccgctacgctaatcacctgctcgcctatctgcgatcggcatggaaatacagcgaccgcctgtttgcctacggcctgatttctaccaat

SEQ ID NO: 15 - DNA, 498 bp, Unknown
  DNA retrieved from environmental DNA; Amylase gene; am162
atgataggttacgagatatttgtgaggtcctttgcggactcaaatgatgacggaattggggatttcaaaggcatcgcccagaaagtcgactatttcaagatgctcggcgtagacttaatctggttaacgccgcacttcaagtcaccaagttaccacggttacgacataatcgactactttgacacgaatgtctcgttcggaacacttgcagattttagagatatggtcgacaagctgcatgcgaatggaataaaaattgtcatcgacctgccgttcaaccacgtctcagacaggcacccatggttcaaagccgctatgaacggcgaaaaaccgtatgttgattacttcctctgggcgcagccgcacttcaatttgaaagaaaaaagacactgggacgaagaattgctttggcacacgagaaatggcaagacatactacggcgtgttcggtggttcttcgcccgacttgaattatgaaaaccccgaagttgtgcaaaat

SEQ ID NO: 16 - DNA, 299 bp, Unknown
  DNA retrieved from environmental DNA; Amylase gene; am163
cgtgagacgccgattcttcagtggttccagaccgattaccgcaccattttgcagcgtctgcctgaagtagtgcaggcgggctacggcgcgatttacctcccctcgcccgtcaagtctggcggtggggggttcagcacgggctacaaccccttcgatctgtttgacttgggcgaccgcttccagaaaggcactgtacgaacgcaatacggcacgactcaggaactgatagagctgattcgccttgcgcagcgactggggctggaggtctattgcgacttggtgaccaaccatgcggacaa

SEQ ID NO: 17 - DNA, 530 bp, Unknown
  DNA retrieved from environmental DNA; Amylase gene; am164
atgagtgataccgaaaaacctcgccgcacccgccgtaaacaggtggcgaatactgatgagccttccacgacagtgacggcctcgaccacggatgcaccaaccgcaaccattgaggaaccttcggcggctgctcgtgctatgatgaccagtatcctcagcgaggatgatatttatctgttcaaccagggcacccattaccgcttgtacgacaaatttggtgctcagccggtggtgctggaaggtgtaccgggcacctattttgcggtttgggcaccaaatgccgagtatgtggccgtgatcggcgactggaataactgggacgccggtgccaacccgctccggcagcgcggcttttcgggtgtgtgggagggatttatcccccacgtcggtaaaggcatgcgctacaagttccacatcgcctcgcgctactacggctatcgcgaagacaagacagatcccttcggcacctacttcgaggtcgcaccgcagacggctgccattatctgggatcgcgattacacctggtcgga

SEQ ID NO: 18 - DNA, 570 bp, Unknown
  DNA retrieved from environmental DNA; Amylase gene; am170
agtagtcttccgttcggtccggtgcaccattcaaccgcacgtgcccaaacctcatcaccacgtaccgtatttgttcatctctttgaatggaagtggacggacattgcccaggaatgcgagaactttctggggccacgcggctttgcggcagtgcaggtgtcgccaccgcaagagcacgcgattgttgccggttatccgtggtggcaacggtatcaaccggtcagttatcaattgaccagtcgtagcgggacacgggctgaattcgccaatatggttgcccgttgcaaagcggtcggtgttgacatttatgttgatgcggtcatcaatcatatgaccggcgtcggcagcggtgtcggatcggctggctcaacgtatagcccgtacaactatccgggcatctatcaatatcaggattttcaccactgcggcagaaatggcaacgatgacatccagaattatggtgatcggtacgaagttcagaactgcgaactggtgaatcttgccgatctcgataccggatcatcgtatgtgcgggatcgcttagctgcctatttgaacgatctcatcatg

SEQ ID NO: 19 - DNA, 685 bp, Unknown
  DNA retrieved from environmental DNA; Amylase gene; am173
ctgtttccagaaaaactgggagcgcaccccacagaaatagacggcgttaagggtgtttattttgccgtttgggctcccaatgcacgtaacgtttccgtgattggcgatttcaatcagtgggatggacgcaaacatcagatgcgtaaaggacaaactggggtttgggaattgtttattcctgaacttggggtaggagaacattacaaatacgaaatcaaaaatctagaaggtcacatttacgaaaaatctgacccctacggtttccaacaagaacctcgtcccaaaacagcatcgattgtcactgacttaaatagctatcagtggaacgacgaagattggatggagcagcggcgtcacacctatcctctgactcaacccatctcagtttacgaagtacatttaggttcttggttacacgcctctagcgcagaaccacctagactacctaatggggaaaccgagcctgtcgttcctgtttctgaacttaatcctggtgcgcgttttctgacttatcgagagctagcagacaggttaatcccctacgtcaaagatttgggctatacccatgtggaattattgcctatcgctgaacatccctttgatggttcttggggttaccaagtcacaggctattacgcccctacttcccgttatggtagcccagaagattttatgtattttgttg

SEQ ID NO: 20 - DNA, 1428 bp, Unknown
  DNA retrieved from environmental DNA; Amylase gene; am159-G
gtgacctggtacgagggcgctttcttctaccagatctttcccgaccgctacttccgggctggccctttcggaaagccagtcccggtaggggctttggaaccctgggaaacacccccctcccttaggggctkcaagggcgggaccctctggggcatagcggagaaaatcccctacctcaaggacctgggggtggaagccctttacctgaaccccgtcttcgcctccaccgccaaccaccggtaccacaccacggactatttccaggtggatcccctcctgggggggaacgtggccctaaggcacctcctggaagtcgcccacgcccacggcatgcgggtcatcctggacggggtcttcaaccacacgggtaggggcttttttgccttccagcaccttctggaaaacggagaacaaagcccctaccgggactggtaccacgtgaagggttttcccctaaacccctatagccgccaccccaactacgaggcctggtggggcaatcctgagcttcccaarctccgggtggaaaccccggcggtgcgggagtacctcctggaggtggcggagcactggatccgcttcggcgcggatggctggcggctggacgtgcccaacgagatccccgaccccgagttctggcgggccttccgcaggagggtgaagggggcgaacccggaggcctacctcgtgggggagatctgggaggaggccgaggcctggctccagggggacatctttgacggggtgatgaactaccccctcgcccgggcggttctaggcttcgtgggaggggaggccctggaccgggagcttgccgcccgctcgggcctagggcgggtggaacccctccaggccctggccttcagccaccgcctcgaggaccttttcggccggtatccctgggcggcggtcctggcccagatgaacctcctcacctcccacgacaccccgaggctcctctccctcctccggggggacgtggcccgggcgcgcctggccctgagcctcctcttcctcctcccgggaaaccccacggtctactacggggaggaagtggggatggagggcggccctgaccccgagaaccgcggggggatggtgtgggaggaagggcgctggcggggggagctccgcgaggcggtgaggaggatggcgaggctgcgccaggcccatcccgagctccgcaccgccccctaccggcgggtctacgcccaggaccggcacctggccttcacccgcgggccctacctggcggtggtgaacgccagcgaccgccccttccggcaggaccttcccctgcacggcgtcttcccccgggggggtgaggccctggacctcctctcgggggcccgggccaagctccaggggggaaggctcctgggccccgagctgccccccttcgccctcgccctgtggcaggaggtgtga

SEQ ID NO: 21 - DNA, 1365 bp, Unknown
  DNA retrieved from environmental DNA; Amylase gene; am162-G
atgataggttacgagatatttgtgaggtcctttgcggactcaaatgatgacggaattggggatttcaaaggcatcgcccagaaagtcgactatttcaagatgctcggcgtagacttaatctggttaacgccgcacttcaagtcaccaagttaccacggttacgacataatcgactactttgacacgaatgtctcgttcggaacacttgcagattttagagatatggtcgacaagctacatgcgaatggaataaaaattgtcatcgacctgccgttcaaccacgtctcagacaggcacccatggttcaaagccgctatgaacggcgaaaaaccgtatgttgattacttcctctgggcgcagccgcacttcaatttgaaagaaaaaagacactgggacgaagaattgctttggcacacgagaaatggcaagacatactacggcgtgttcggtggttcttcgcccgacttgaattatgaaaaccccgaagttgtgcaaaaatcactcgagatagttgaattctggctcaagcagggcgttgatggattcagatttgatgcggcaaagcacatatacgactacgatatcaaagaaggcaaattcagatacgaccacgaaaagaatgtcgcctattggcaactcgttatggacagagcaaggcaaatcaaaggagaagatgtattcgcagttacggaagtctgggacgatcctgaaatcgttgacaggtacgctaagacaatcggctgttcgttcaacttctacttcacagaagccataagagaatcgatgcagcacggagcggtgtacaaaatcgtcgactgctttcagagaacactcacgaaaaagccatacctgccaagcaacttcacaggcaaccacgacatgcacagactggctcagctactaccacatgaagagcagagaaaagtcttcttcggactgctcatgacaacacccggcgttccgttcatatactacggcgatgagctcggaatgaaggggcagtacgactccacattcacagaagacgttatagaaccattcccatggtacgcttcgctatctggcgagggccaagcgttctggaaggctgtaaggttcaacagggcattcaccggtgcttctgttgaggaacacctgaaccgcgaggacagtctgctcaaagaagttattaactggacaaagttcaggaaagaaaacgactggctcacaaacgcatgggtagagcacgtaacgcacaacacgttcacaatcgcttatacggttacagacggcgacaacggattcagagtttatgtgaacatagctggccaccacgagaccttcgaaggagtaagtctcaaagcgtacgaagttaaggttctctga

SEQ ID NO: 22 - DNA, 2034 bp, Unknown
  DNA retrieved from environmental DNA; Amylase gene; am164-G
atgagtgataccgaaaaacctcgccgcacccgccgtaaacaggtggcgaatactgatgagccttccacgacagtgacggcctcgaccacggatgcaccaaccgcaaccattgaggaaccttcggcggctgctcgtgctatgatgaccagtatcctcagcgaggatgatatttatctgttcaaccagggcacccattaccgcttgtacgacaaatttggtgctcagccggtggtgctggaaggtgtaccgggcacctattttgcggtttgggcaccaaatgccgagtatgtggccgtgatcggcgactggaataactgggacgccggtgccaacccgctccggcagcgcggcttttcgggtgtgtgggagggatttatcccccacgtcggtaaaggcatgcgctacaagttccacatcgcctcgcgctactacggctatcgcgaagacaagacagatcccttcggcacctacttcgaggtcgcaccgcagacggctgccattatctgggatcgcgattacacctggtcggatcaacagtggatgagcgaacgggggcagcggcagcgcctcgatgcgccgatctccatctacgaagtgcatttgggatcgtggcggcgcaaaccggaagaggataaccgtccgctcaattaccgtgaactggcccacgagctggtcgagcatgtgaaagattgtggctttacccacgttgagctgttaccggtcaccgagcatcccttctacggttcctgggggtatcaatcgacgggtttgttcgcgccgaccagccggtacggaacgccgcaagacttcatgtattttgtggattatctgcatcaaaacgggattggggtgatcctcgattgggtgcccagccacttcccgaccgacggtcatgggctggcctacttcgatggtacccatctctacgaacacgccgatccgcgtaaaggctaccatcccgactggggaagctatatttacaactatggtcggaacgaggtacgaagcttcctgatcagctcggcgctctgctggctggataagtttcacattgacgggatacgggttgatgcggttgcgagcatgctctatctcgactattcgcgccgagccggcgagtggattcccaacgaatacggtgggaacgaaaatctggaggcgattagcttcctgcgcgaattgaacacccagatttacaagtactaccctgatgtgcagacaattgccgaggagagcacagcctggccgatggtatcgcgaccggtctacgttggtggattgggcttcggcttcaagtgggacatgggctggatgcacgataccctgcagtatttccggcgcgatccgatctaccggcgctttcatcacaacgaattgaccttccgtggcctctacatgttcagcgagaactacgtgctaccactctcgcacgatgaggtcgttcacggcaaagggtcactgctcgacaagatggccggcgatgtctggcaaaagtttgccaacctgcgcctgctctacagctatatgtttgctcaacccggtaaaaaactgctcttcatgggtggtgaattcggacagtggcgcgaatggtcacacgacaccagcctggactggcacttactgatgtttccctcccatcagggcgtacaacgattgattggcgatcttaaccgtctctaccgtactgagccggccttgcacgaactggactgtgatccacgtgggtttgagtggatcgatgccaatgatgccgatgccagcgtctacagctttctgcgcaagagccgctacggcgagcaaattctgatcgtgatcaatgccacgccggtcgtgcgtgaggattaccgaattggggtaccggtgggtggctggtggcgtgaattgtttaacagcgactcggagtattattggggaagtgggcaaggcaatgccggcggcgtgatggccgaagcaattccaacccatggccgggatttttcgttgcgactgcgcctgccgcccctgggtgcgctcttcctgaaacctgccggctaa

SEQ ID NO: 23 - DNA, 1863 bp, Unknown
  DNA retrieved from environmental DNA; Amylase gene; am170-G
tcattccactactcactgttgttgagtctggtcagcgttggccgcttcctggagcaaaggagcctgtttatgcccggcactcgctttccctcgcttcgtcggctcgtcctcgttgtcgcccttctcatggtggtaagtagtcttccgttcggtccggtgcaccattcaaccgcacgtgcccaaacctcatcaccacgtaccgtatttgttcatctctttgaatggaagtggacggacattgcccaggaatgcgagaactttctggggccacgcggctttgcggcagtgcaggtgtcgccaccgcaagagcacgcgattgttgccggttatccgtggtggcaacggtatcaaccggtcagttatcaattgaccagtcgtagcgggacacgggctgaawtcccccatatggttgcccgttgcaaagcggtcggtgttgacatttatgttgatgcggtcatcaatcatatgaccggcgtcggcagcggtgtcggatcggctggctcaacgtatagcccgtacaactatccgggcatctatcaatatcaggattttcaccactgcggcagaaatggcaacgatgacatccagaattatggtgatcggtacgaagttcagaactgcgaactggtgaatcttgccgatctcgataccggatcatcgtatgtgcgggatcgcttagctgcctatttgaacgatctcatcagtctgggagttgccggttttcggattgacgcagctaaacacattgctgccggggatattgccgcaattttatcccgtgtgaatgggagtccgtacatttaccaggaagtgatcggtgcggctggcgaaccgattacaccgtgggaatacacaaataatggtgatgtcactgaatttaagtatagcaacgagatcgggcgggtctttttgaatggtaagctggcatggctgagtcagtttggcgaagcctgggggatgctgccaagcgacaaagcgattgtcttcgttgataatcacgacaaccagcgcgggcatggcggtggtgggactgtggtcacatacaagaatggtgtgctgtacgatctggcaaacgtgtttatgctagcgtggccgtatgggtacccccaggtgatgtcaagttatgagtttagcaatgattttcaagggccaccgagtgatgcgaacggcaacacgcgcagcgtctatgttaacggncagcccaattgctttggcgaatggaaatgcgagcatcgctggcgaccaattgcgaatatggtagcgttccgcaatgccacagcgagtacattcagtgtgagtgattggtggagtaacggcaacaaccagatcgcctttggtcgtggcgataaagggtttgtcgttatcaatcgtgaggatacaacgctgaatcgcacgtttcagacgagtatggcgcctggggtctactgcaatgtgattgttgccgattttacaaacggtacgtgcagtgggcaaaccgtcaccgtggacagtaatcgacggataacggtctctattccgcctttcagtgctcttgccatccatgtaggagcgaagttgtctacgcaaccggcaactgttgcggttactttcaacgtgaatgcgacgacctactgggggcagaacgtgtttgtggttgggaatatcccgcaattgggcaactggaacccggcgcaggctgtgcccctttcagcggctacgtatccggtctggagtggtaccgttaatctgccggcaaataccaccatcgaatacaagtacattaagcgtgacggatcaaatgtggtgtgggagtgttgtaataatcgcgttattacgacgccaggtagtggctcgatgacgctgaatgagacgtggcgtccgtga

SEQ ID NO: 24 - DNA, 405 bp, Unknown
  DNA retrieved from environmental DNA; Amylase gene; am80
accgatctgggagtctcggcactgtacctcaatcctatcttccgagcgccgtcgaaccacaaatacgatgtcgaagactataccagcattgaccctcacctgggaggtgaagcagggttactcctcttacgcgaggtactcgacgagcgagccatgaagctggtgcttgacatcgtcccgaaccattgtggagtgacccacccgtggtttgtcgctgcccaggccaacccacgatcaccaacagccgagttcttcatgttccgtcgtcatcccgacggctacgagagctggctgggggtcaagaccctgcccaaactcaattaccgcagtgtccgcctccgcgacgtaatgtacgcaggccaggatgcgattatgcgctactggttgcgaccaccctatcggatc

SEQ ID NO: 25 - DNA, 474 bp, Unknown
  DNA retrieved from environmental DNA; Amylase gene; am81
gccgattgtttgattagcgattacagtgatcgctatcaggtccagtattgtcagttagccggcctgccagacctcgataccggtaagagcactgtgcagacgaagctgcgtgcttacctgcaagccctgctcaatgccggtgtcaaaggcttccgcattgatgctgccaagcacatggccgcgcacgaggtcggtgccattctcgatgggctgaccctccccggcggcggtcgtccgtacatcttcagtgaagtcattgacatggatcccaatgagcggatacgcgattgggaatacacgccttacggagacgtcaccgagtttgcctacagtattagcgtgatcgggaataccttcaattgtggtggatcgctcagcaatctgcaaaacttcaccacgaacctactgccctcgcacttcgcccagattttcgttgacaaccacgacacccagcggggcaagggcgaattcgtt

SEQ ID NO: 26 - DNA, 222 bp, Unknown
  DNA retrieved from environmental DNA; Amylase gene; am82
ggcgagattgttgatccctccgatgttcaaatggcctttgccgggcaactggatggcgcgctagactttatcttgctggaaggtttgcgtcaggctatcgcatttgggcgctggaatggctttcaacttgcctcgtttttagaacggcaccagatttattttccggaagatttctctcgtccatcgttcttggacaaccacgacacccagcggggcaagggc

SEQ ID NO: 27 - DNA, 474 bp, Unknown
  DNA retrieved from environmental DNA; Amylase gene; am103
gattttcacgccgattgtttgattagcgattacagtgatcgctatcaggtccagtattgtcagttagccggcctgccagacctcgataccggtaagagcactgtgcagacgaagctgcgtgcttacctgcaagccctgctcaatgccggtgtcaaaggcttccgcattgatgctgccaagcacatggccgcgcacgaggtcggtgccattctcgatgggctgaccctccccggcggcggtcgtccgtacatcttcagtgaagtcattgacatggatcccaatgagcggatacgcgattgggaatacacgccttacggagacgtcaccgagtttgcctacagtattagcgtgatcgggaataccttcaattgtggtggatcgctcagcaatctgcaaaacttcaccacgaacctactgccctcgcacttcgcccagattttcgttgacaaccacgacacccagcggggcaagggc

SEQ ID NO: 28 - DNA, 263 bp, Unknown
  DNA retrieved from environmental DNA; Aminocylase/Amidohydrolase; EAA10
atgaaactgatagacagcattgtgcaaaacacaccgacgatcgcggcggtgcgacgcgatctgcacgcccaccccgaattgtgttttgaggaaaaccgcacggccgacaaggtcgcatccaagctcgcggagtggggcatcccgttccatcgtggccttgcgactactggcgtggtgggcatcatccagtcgggcacttctgacagagccattggcttgcgcgctgatatggacgcgttgccgatgcaagaggtcaatacctt

SEQ ID NO: 29 - DNA, 252 bp, Unknown
  DNA retrieved from environmental DNA; Aminocylase/Amidohydrolase; EAA11
atgaaccttattgactccattgtttccagcgccgcgtccattgcagccgtccgccgcgatctacatgcccatccggagctgtgttttaaggaagtgcacacttccgatgtcgtggcacagcggctgaccgattggggtatcccgattcaccgcggtctcggcaccacgggcgtcgtgggcatcatcaaagcgggcacctccgaccgtgctattgccttgcgagccgatatggacgcgcttcccatgcaggaa

SEQ ID NO: 30 - DNA, 480 bp, Unknown
  DNA retrieved from environmental DNA; Aminocylase/Amidohydrolase; EAA12
atcacaccggaaggccatattttgggtcgttacagcaagaaccagcccttcagcctcggcggtgaaagcaccgtgcataccgctggcaaaggcgtgaccgtcgtcgagtggcagggcatcaagattgcaccgctcatctgctatgatctgcgctttccggagctcgctcgcgaggccgtgaaggccggcgccgagctgctcgtcttcatcgccgcgtggccgatcaaacgcgtgcagcattggatcacgctgctgcaagcccgtgcgatcgaaaacctcgcgttcgtcatcggcgtgaaccaatgcggcaccgatccgagcttcacatatcccgggcgcagcctcgtcgtcgatccgcacggcgtcatcatcgccgatgcgggcgatcacgagcacgtcctgcgtgccgagatcgatcccgccatcctccacgcctggcgcagccagttccccgccttgcgtgacgcgggaatcgcgtcg

SEQ ID NO: 31 - DNA, 292 bp, Unknown
  DNA retrieved from environmental DNA; Aminocylase/Amidohydrolase; EAA13
atgaaactgatccccgaaatccaggccgctcaaggcgagatacaaaccctccgacgaacgattcacgcccacccagaactgcgttacgaagaaactcagacatccgacctggtcgcgaagagtttgagcgactggggtatcgaggtgcatcgtgggctcggcaaaaccggggttgtgggcattctgaagcgtggcagcagcgagcgggcaataggcctgagggccgacatgaacgccctgccgatccacgaattgaacagcttcgagcatcgttcacgccacgaaggaatgt

SEQ ID NO: 32 - DNA, 27 bp, Artificial Sequence
  misc_feature (1)...(27): n = A,T,C or G
cattgccgtatggccatcrtgnccrca

SEQ ID NO: 33 - DNA, 23 bp, Artificial Sequence
  misc_feature (1)...(23): n = A,T,C or G
ggccgtgtggcctcrtgnccrca

SEQ ID NO: 34 - DNA, 23 bp, Artificial Sequence
  Adaptor oligonucleotide
aagggtgccaacctcttcaaggg

SEQ ID NO: 35 - DNA, 20 bp, Artificial Sequence
  Primer used to amplify environmental DNA
cttgaagaggttggcaccct

SEQ ID NO: 36 - DNA, 40 bp, Artificial Sequence
  misc_feature (1)...(40): n = A,T,C or G
gatatttaatatgtttagctgcatcaattckraanccrtc

SEQ ID NO: 37 - DNA, 24 bp, Artificial Sequence
  misc_feature (1)...(24): n = A,T,C or G
ggcggcgtcgatcckraanccrtc

SEQ ID NO: 38 - DNA, 37 bp, Artificial Sequence
  misc_feature (1)...(37): n = A,T,C or G
gatcaacttaattagcaacatccattckccanccrtc

SEQ ID NO: 39 - DNA, 24 bp, Artificial Sequence
  misc_feature (1)...(24): n = A,T,C or G
gccccgctgggtgtcrtgrttntc

SEQ ID NO: 40 - DNA, 30 bp, Artificial Sequence
  misc_feature (1)...(30): n = A,T,C or G
gcatgttatgctggatgcagtnttyaayca

SEQ ID NO: 41 - DNA, 36 bp, Artificial Sequence
  misc_feature (1)...(36): n = A,T,C or G
aaatgtgcaagtgtatatggattttgtnytnaayca

SEQ ID NO: 42 - protein, 48 aa, Unknown
  Polypeptide encoded by DNA retrieved from environmental DNA; Aminoacylase/Amidohydrolase polypepetides; EAA1
NRGMGTTGVVGIVKAGTSERAIALRADMDALPTQEFNTFEHASQHPGK

SEQ ID NO: 43 - protein, 59 aa, Unknown
  Polypeptide encoded by DNA retrieved from environmental DNA; Aminoacylase/Amidohydrolase polypeptides; EAA2
VVLQFTGRRFTHRGLGTTGVVGIVKAGTSERALALRADMDALPMQECNSFAHTSQYPGK

SEQ ID NO: 44 - protein, 90 aa, Unknown
  Polypeptide encoded by DNA retrieved from environmental DNA; Aminoacylase/Amidohydrolase polypeptides; EAA3
LHELTAFRRDLHVHPELGFEEVYTSGRVAETLRLCGVDEVHTQIGKTGVVAVIKGKRQSSGKMMGLRADMDALPMAEHNEFTWKSAKSGL

SEQ ID NO: 45 - protein, 120 aa, Unknown
  Polypeptide encoded by DNA retrieved from environmental DNA; Aminoacylase/Amidohydrolase polypepetides; EAA4
LKPAPPQCYSEMALLSRRRSMIQFPFRRCRMLQKAQEIQEPLVAWRREFHTYPELGFRESRTAARVAEILTGLGYRVRTGVGRTGVVAERGEGHPIIAVRADMDALPIQEANDVPYASQH

SEQ ID NO: 46 - protein, 99 aa, Unknown
  Polypeptide encoded by DNA retrieved from environmental DNA; Aminoacylase/Amidohydrolase polypeptides; EAA5
LPELLDQADAMRALRRDIHAHPELCFQEVRTSDLIAKTLQSWGIEVHTGLGTTGVVGVIKGRPGKRAIGLRADIDALPMTEHNTFAHASRHACKTTAQG

SEQ ID NO: 47 - protein, 81 aa, Unknown
  Polypeptide encoded by DNA retrieved from environmental DNA; Aminoacylase/Amidohydrolase polypeptides; EAA6
GDALTERVGEFIQLRRDIHRHPELAFEEHRTSELVAAKLESWGYAVRRGLGGTGVVGVLKRGHSQRSLGIRADMDALPIQE

SEQ ID NO: 48 - protein, 101 aa, Unknown
  Polypeptide encoded by DNA retrieved from environmental DNA; Aminoacylase/Amidohydrolase polypeptides; EAA7
PSLPPSVLPELLDQADAMRALRRDIHAHPELCFQEVRTSDLIAKTLQSWGIEVHTGLGTTGVVGVIKGRPGKRAIGLRADIDALPMTEHNTFAHASRHAGR

SEQ ID NO: 49 - protein, 52 aa, Unknown
  Polypeptide encoded by DNA retrieved from environmental DNA; Aminoacylase/Amidohydrolase polypeptides; EAA8
GIPLHRGMGTTGVVGIVKSGTSDRAIGLRADMDALPMAEANTFAHASTHPGK

SEQ ID NO: 50 - protein, 92 aa, Unknown
  Polypeptide encoded by DNA retrieved from environmental DNA; Aminoacylase/Amidohydrolase polypeptides; EAA9
ITEFHPELTAFRRDLHVHPELGFEEVYTSGRVAEGLRLCGVDEVHTQIGKTGVVAVIKGKRQTSGKMIGLRADMDALPMAEHNEFTWKSAKT

SEQ ID NO: 51 - protein, 99 aa, Unknown
  Polypeptide encoded by DNA retrieved from environmental DNA; Amylase polypeptide; am27
MVARCKAVGVDIYVDAVINHMTGVGSGVGSAGSTYSPYNYPGIYQYQDFHHCGRNGNDDIQNYGDRYEVQNCELVNLADLDTGSSYVRDRLAAYLNDLI

SEQ ID NO: 52 - protein, 124 aa, Unknown
  Polypeptide encoded by DNA retrieved from environmental DNA; Amylase polypeptide; am80
ICLAASIRKPSNHKYDVEDYTSIDPHLGGEAGLLLLREVLDERAMKLVLDIVPNHCGVTHPWFVAAQANPRSPTAEFFMFRRHPDDYESWLGVKTLPKLNYRSVRLRDVMYAGQDAIMRYWLRP

SEQ ID NO: 53 - protein, 35 aa, Unknown
  Polypeptide encoded by DNA retrieved from environmental DNA; Amylase polypeptide; am156
RKPEEDNRPLNYRELAHELAEHXKDCGFTHVELLP

SEQ ID NO: 54 - protein, 213 aa, Unknown
  Polypeptide encoded by DNA retrieved from environmental DNA; Amylase polypeptide; am159
TAATSTPTLTITPTTSPIDKPEWWKSAVFYQVFVRXFYDSDGDGIGDFQGLIQKLDYLNDGDPKTNSDLGINAVWLMPVNPSPSYHGYDVTDYYNVNPDYGTMDDFRELIKEAHQRGIKVIIDLVINHTSTQHPWFQQALDPQSPYHNYYIWRDENPGYSGPDGQKVWHRASNGKYYYALFWDQMPDLNFQNPQVTEEIYQIARFWLEDVGVD

SEQ ID NO: 55 - protein, 137 aa, Unknown
  Polypeptide encoded by DNA retrieved from environmental DNA; Amylase polypeptide; am161
YNDNISTAGPFNFLPSPALKVTLVGLGYRLNNQTFYPDYQSEVMGAVSLVRRMFPLANSAGGSGLAWDYWHIMDEGLGSRVNMTNVECNDYISWEDGKVVDRRNLCSTRYANHLLAYLRSAWKYSDRLFAYGLISTN

SEQ ID NO: 56 - protein, 166 aa, Unknown
  Polypeptide encoded by DNA retrieved from environmental DNA; Amylase polypeptide; am162
MIGYEIFVRSFADSNDDGIGDFKGIAQKVDYFKMLGVDLIWLTPHFKSPSYHGYDIIDYFDTNVSFGTLADFRDMVDKLHANGIKIVIDLPFNHVSDRHPWFKAAMNGEKPYVDYFLWAQPHFNLKEKRHWDEELLWHTRNGKTYYGVFGGSSPDLNYENPEVVQN

SEQ ID NO: 57 - protein, 99 aa, Unknown
  Polypeptide encoded by DNA retrieved from environmental DNA; Amylase polypeptide; am163
RETPILQWFQTDYRTILQRLPEVVQAGYGAIYLPSPVKSGGGGFSTGYNPFDLFDLGDRFQKGTVRTQYGTTQELIELIRLAQRLGLEVYCDLVTNHAD

SEQ ID NO: 58 - protein, 176 aa, Unknown
  Polypeptide encoded by DNA retrieved from environmental DNA; Amylase polypeptide; am164
MSDTEKPRRTRRKQVANTDEPSTTVTASTTDAPTATIEEPSAAARAMMTSILSEDDIYLFNQGTHYRLYDKFGAQPVVLEGVPGTYFAVWAPNAEYVAVIGDWNNWDAGANPLRQRGFSGVWEGFIPHVGKGMRYKFHIASRYYGYREDKTDPFGTYFEVAPQTAAIIWDRDYTWS

SEQ ID NO: 59 - protein, 190 aa, Unknown
  Polypeptide encoded by DNA retrieved from environmental DNA; Amylase polypeptide; am170
SSLPFGPVHHSTARAQTSSPRTVFVHLFEWKWTDIAQECENFLGPRGFAAVQVSPPQEHAIVAGYPWWQRYQPVSYQLTSRSGTRAEFANMVARCKAVGVDIYVDAVINHMTGVGSGVGSAGSTYSPYNYPGIYQYQDFHHCGRNGNDDIQNYGDRYEVQNCELVNLADLDTGSSYVRDRLAAYLNDLIM

SEQ ID NO: 60 - protein, 228 aa, Unknown
  Polypeptide encoded by DNA retrieved from environmental DNA; Amylase polypeptide; am173
LFPEKLGAHPTEIDGVKGVYFAVWAPNARNVSVIGDFNQWDGRKHQMRKGQTGVWELFIPELGVGEHYKYEIKNLEGHIYEKSDPYGFQQEPRPKTASIVTDLNSYQWNDEDWMEQRRHTYPLTQPISVYEVHLGSWLHASSAEPPRLPNGETEPVVPVSELNPGARFLTYRELADRLIPYVKDLGYTHVELLPIAEHPFDGSWGYQVTGYYAPTSRYGSPEDFMYFV

SEQ ID NO: 61 - protein, 563 aa, Unknown
  Polypeptide encoded by DNA retrieved from environmental DNA; Amylase polypeptide; am159-G
MKLTRLRHITVLIIILSLLGACTTPQKPSNEGAAATSTPTLTITPTTSPIDKPEWWKSAVFYQVFVRSFYDSDGDGIGDFQGLIQKLDYLNDGDPKTNSDLGINAVWLMPVNPSPSYHGYDVTDYYNVNPDYGTMDDFRELIKEAHQRGIKVIIDLVINHTSTQHPWFQQALDPQSPYHNYYIWRDENPGYSGPDGQKVWHRASNGKYYYALFWDQMPDLNFQNPQVTEEIYQIARFWLEDVGVDGFRIDAAKHLIEEGTDQENTGLTHEWFASFYQYYKSLNPQAVTVGEVWSNSFEAVRYVRNQEMDMVFNFDLARSIXTXINNRNAVSLSNTLTFEXRLFPKGSMGIFXTNHDQDRVMTVLMNDEQKARLXAAVYXTSPGVPFIYYGEEIGLTGQGDHRNIRTPMHWSAERMAGFTSGTPWLFPKMDYAEKNVEDQLEDPNSLLRFYMDLLRIRSQSKALQSGELSALSSSSSSILAYARVSQNEQVLIVLNLGNQPQERVTLHSVEGLNPGTYRLSPLLGGQVNTTIIVEPDGALQEFEFPATISANEVLIYQLINSTE

SEQ ID NO: 62 - protein, 454 aa, Unknown
  Polypeptide encoded by DNA retrieved from environmental DNA; Amylase polypeptide; am162-G
MIGYEIFVRSFADSNDDGIGDFKGIAQKVDYFKMLGVDLIWLTPHFKSPSYHGYDIIDYFDTNVSFGTLADFRDMVDKLHANGIKIVIDLPFNHVSDRHPWFKAAMNGEKPYVDYFLWAQPHFNLKEKRHWDEELLWHTRNGKTYYGVFGGSSPDLNYENPEVVQKSLEIVEFWLKQGVDGFRFDAAKHIYDYDIKEGKFRYDHEKNVAYWQLVMDRARQIKGEDVFAVTEVWDDPEIVDRYAKTIGCSFNFYFTEAIRESMQHGAVYKIVDCFQRTLTKKPYLPSNFTGNHDMHRLAQLLPHEEQRKVFFGLLMTTPGVPFIYYGDELGMKGQYDSTFTEDVIEPFPWYASLSGEGQAFWKAVRFNRAFTGASVEEHLNREDSLLKEVINWTKFRKENDWLTNAWVEHVTHNTFTIAYTVTDGDNGFRVYVNIAGHHETFEGVSLKAYEVKVL

SEQ ID NO: 63 - protein, 677 aa, Unknown
  Polypeptide encoded by DNA retrieved from environmental DNA; Amylase polypeptide; am164-G
MSDTEKPRRTRRKQVANTDEPSTTVTASTTDAPTATIEEPSAAARAMMTSILSEDDIYLFNQGTHYRLYDKFGAQPVVLEGVPGTYFAVWAPNAEYVAVIGDWNNWDAGANPLRQRGFSGVWEGFIPHVGKGMRYKFHIASRYYGYREDKTDPFGTYFEVAPQTAAIIWDRDYTWSDQQWMSERGQRQRLDAPISIYEVHLGSWRRKPEEDNRPLNYRELAHELVEHVKDCGFTHVELLPVTEHPFYGSWGYQSTGLFAPTSRYGTPQDFMYFVDYLHQNGIGVILDWVPSHFPTDGHGLAYFDGTHLYEHADPRKGYHPDWGSYIYNYGRNEVRSFLISSALCWLDKFHIDGIRVDAVASMLYLDYSRRAGEWIPNEYGGNENLEAISFLRELNTQIYKYYPDVQTIAEESTAWPMVSRPVYVGGLGFGFKWDMGWMHDTLQYFRRDPIYRRFHHNELTFRGLYMFSENYVLPLSHDEVVHGKGSLLDKMAGDVWQKFANLRLLYSYMFAQPGKKLLFMGGEFGQWREWSHDTSLDWHLLMFPSHQGVQRLIGDLNRLYRTEPALHELDCDPRGFEWIDANDADASVYSFLRKSRYGEQILIVINATPVVREDYRIGVPVGGWWRELFNSDSEYYWGSGQGNAGGVMAEAIPTHGRDFSLRLRLPPLGALFLKPAG

SEQ ID NO: 64 - protein, 597 aa, Unknown
  Polypeptide encoded by DNA retrieved from environmental DNA; Amylase polypeptide; am170-G
MPGTRFPSLRRLVLVVALLMVVSSLPFGPVHHSTARAQTSSPRTVFVHLFEWKWTDIAQECENFLGPRGFAAVQVSPPQEHAIVAGYPWWQRYQPVSYQLTSRSGTRAEXPHMVARCKAVGVDIYVDAVINHMTGVGSGVGSAGSTYSPYNYPGIYQYQDFHHCGRNGNDDIQNYGDRYEVQNCELVNLADLDTGSSYVRDRLAAYLNDLISLGVAGFRIDAAKHIAAGDIAAILSRVNGSPYIYQEVIGAAGEPITPWEYTNNGDVTEFKYSNEIGRVFLNGKLAWLSQFGEAWGMLPSDKAIVFVDNHDNQRGHGGGGTVVTYKNGVLYDLANVFMLAWPYGYPQVMSSYEFSNDFQGPPSDANGNTRSVYVNXQPNCFGEWKCEHRWRPIANMVAFRNATASTFSVSDWWSNGNNQIAFGRGDKGFVVINREDTTLNRTFQTSMAPGVYCNVIVADFTNGTCSGQTVTVDSNRRITVSIPPFSALAIHVGAKLSTQPATVAVTFNVNATTYWGQNVFVVGNIPQLGNWNPAQAVPLSAATYPVWSGTVNLPANTTIEYKYIKRDGSNVVWECCNNRVITTPGSGSMTLNETWRP

SEQ ID NO: 65 - protein, 135 aa, Unknown
  Polypeptide encoded by DNA retrieved from environmental DNA; Amylase polypeptide; am80
TDLGVSALYLNPIFRAPSNHKYDVEDYTSIDPHLGGEAGLLLLREVLDERAMKLVLDIVPNHCGVTHPWFVAAQANPRSPTAEFFMFRRHPDGYESWLGVKTLPKLNYRSVRLRDVMYAGQDAIMRYWLRPPYRI

SEQ ID NO: 66 - protein, 158 aa, Unknown
  Polypeptide encoded by DNA retrieved from environmental DNA; Amylase polypeptide; am81
ADCLISDYSDRYQVQYCQLAGLPDLDTGKSTVQTKLRAYLQALLNAGVKGFRIDAAKHMAAHEVGAILDGLTLPGGGRPYIFSEVIDMDPNERIRDWEYTPYGDVTEFAYSISVIGNTFNCGGSLSNLQNFTTNLLPSHFAQIFVDNHDTQRGKGEFV

SEQ ID NO: 67 - protein, 74 aa, Unknown
  Polypeptide encoded by DNA retrieved from environmental DNA; Amylase polypeptide; am82
GEIVDPSDVQMAFAGQLDGALDFILLEGLRQAIAFGRWNGFQLASFLERHQIYFPEDFSRPSFLDNHDTQRGKG

SEQ ID NO: 68 - protein, 158 aa, Unknown
  Polypeptide encoded by DNA retrieved from environmental DNA; Amylase polypeptide; am103
DFHADCLISDYSDRYQVQYCQLAGLPDLDTGKSTVQTKLRAYLQALLNAGVKGFRIDAAKHMAAHEVGAILDGLTLPGGGRPYIFSEVIDMDPNERIRDWEYTPYGDVTEFAYSISVIGNTFNCGGSLSNLQNFTTNLLPSHFAQIFVDNHDTQRGKG

SEQ ID NO: 69 - protein, 87 aa, Unknown
  Polypeptide encoded by DNA retrieved from environmental DNA; Aminoacylase/amidohydrolase polypeptides; EAA10
MKLIDSIVQNTPTIAAVRRDLHAHPELCFEENRTADKVASKLAEWGIPFHRGLATTGVVGIIQSGTSDRAIGLRADMDALPMQEVNT

SEQ ID NO: 70 - protein, 84 aa, Unknown
  Polypeptide encoded by DNA retrieved from environmental DNA; Aminoacylase/Amidohydrolase polypeptides; EAA11
MNLIDSIVSSAASIAAVRRDLHAHPELCFKEVHTSDVVAQRLTDWGIPIHRGLGTTGVVGIIKAGTSDRAIALRADMDALPMQE

SEQ ID NO: 71 - protein, 160 aa, Unknown
  Polypeptide encoded by DNA retrieved from environmental DNA; Aminoacylase/Amidohydrolase polypeptides; EAA12
ITPEGHILGRYSKNQPFSLGGESTVHTAGKGVTVVEWQGIKIAPLICYDLRFPELAREAVKAGAELLVFIAAWPIKRVQHWITLLQARAIENLAFVIGVNQCGTDPSFTYPGRSLVVDPHGVIIADAGDHEHVLRAEIDPAILHAWRSQFPALRDAGIAS

SEQ ID NO: 72 - protein, 97 aa, Unknown
  Polypeptide encoded by DNA retrieved from environmental DNA; Aminoacylase/Amidohydrolase polypeptides; EAA13
MKLIPEIQAAQGEIQTLRRTIHAHPELRYEETQTSDLVAKSLSDWGIEVHRGLGKTGVVGILKRGSSERAIGLRADMNALPIHELNSFEHRSRHEGM